(19) United States
(12) Patent Application Publication
(10) Pub. No.: US 2025/0375295 A1
(43) Pub. Date: Dec. 11, 2025

(54) Tissue Fixation Device With Enhanced Tissue Protection

(71) Applicant: Evalve, Inc., Santa Clara, CA (US)

(72) Inventors: Chad J. Abunassar, Alameda, CA (US); Thomas R. Yssels, Mountain View, CA (US); Clara Park, Santa Clara, CA (US); Grant Glaze, San Francisco, CA (US); Hugues A. Gervais, Paris (FR); Anna Maria Snell, Belmont, CA (US)

(73) Assignee: Evalve, Inc., Santa Clara, CA (US)

(21) Appl. No.: 19/221,975

(22) Filed: May 29, 2025

Related U.S. Application Data
(60) Provisional application No. 63/656,130, filed on Jun. 5, 2024.

(51) Int. Cl. A61F 2/24

(52) U.S. Cl. CPC A61F 2/2454; A61F 2220/0016; A61F 2250/0008

(57) ABSTRACT

A fixation device includes first and second distal elements and first and second proximal elements. The first and second proximal elements include a first elongate arm and a second elongate arm, respectively. The first and second elongate arms each include first and second side edges and plurality of frictional elements defining a plurality notches in the first and second side edges. The first and second proximal elements also each include first and second guard rails extending adjacent to and along the first and second side edges, respectively.

### CROSS-REFERENCE TO RELATED APPLICATIONS

This application claims the benefit of the filing date of U.S. Provisional Patent Application No. 63/656,130, filed Jun. 5, 2024, the disclosure of which is hereby incorporated herein by reference.

### BACKGROUND

The cardiac cycle is divided into two phases-diastole and systole. Diastole is generally characterized by the muscular relaxation of the heart and the filling of its chambers with blood. On the other hand, systole is generally characterized by the muscular contraction of the ventricles which pumps blood from the ventricles to the arteries. During ventricular systole, ventricular pressure increases relative to atrial pressure resulting in the closure of the mitral valve and the tricuspid valve. The mitral valve separates the left atrium from the left ventricle, and the tricuspid valve separates the right atrium from the right ventricle. These valves operate as check valves preventing blood from flowing back into the atria during ventricular contraction. However, valvular insufficiency may appear in one or both of these valves which may result in a regurgitative flow back into the atrium across the effected valve. Such regurgitative flow can be in the form of mitral valve regurgitation (“MVR”) and/or tricuspid valve regurgitation (“TVR”). Left untreated, MVR and TVR can lead to severe health consequences, such as progressive heart failure, cardiac arrythmias, pulmonary hypertension, stroke, and endocarditis, to name a few.

MVR and TVR can have a variety of etiologies which typically fall into the categories of degenerative (primary) and functional (secondary) regurgitation. Degenerative valve regurgitation principally occurs due to abnormalities or degeneration of the valve apparatus, such as the valve leaflets, valve annulus, chordae tendineae, and/or papillary muscles. One example of a degenerative valve condition is mitral valve prolapse. Functional valve regurgitation is often a secondary condition that arises from underlying heart conditions or diseases that affect the structure or function of the heart. Examples of conditions that can result in functional regurgitation include dilated cardiomyopathy, ischemic heart disease, pulmonary hypertension, and heart failure. Regardless of the underlying condition precipitating the regurgitative flow, the primary mechanism by which regurgitation occurs is the failure of the valve leaflets to properly and completely seal or coapt during systole which allows a jet of blood to flow back into the atrium between the effected leaflets.

Treatment options for MVR and TVR generally include Guideline-Directed Medical Therapy (“GDMT”), valve replacement, and valve repair. GDMT usually involves the administration of a combination of drugs that treat an underlying heart condition. Valve replacement and repair may include open-heart surgical options and catheter-based options. Catheter-based repair procedures are sometimes referred to as transcatheter edge-to-edge repair (“TEER”).

### BRIEF SUMMARY OF THE DISCLOSURE

In one aspect of the present disclosure, a fixation device may include a first distal element and a second distal element. The fixation device may also include a first proximal element disposed in opposition to the first distal element and moveable relative thereto. The first proximal element may have a first elongate arm which may have first and second side edges and plurality of frictional elements defining a plurality notches in the first and second side edges. The first proximal element may also include a first guard rail extending adjacent to the first side edge and a second guard rail extending along the second side edge. The fixation device may further include a second proximal element disposed in opposition to the second distal element and moveable relative thereto. The second proximal element may have a second elongate arm which may have first and second side edges and plurality of frictional elements defining a plurality notches in the first and second side edges of the second elongate arm. The second proximal element may also having a first guard rail extending adjacent to the first side edge of the second elongate arm and a second guard rail extending along the second side edge of the second elongate arm.

Additionally, the first guard of the respective first and second proximal elements may define a first elongate opening in communication with the plurality of notches formed in the first side edge, and the second guard rail of the respective first and second proximal elements may define a second elongate opening in communication with the plurality of notches formed in the second side edge. The plurality of notches of the first elongate arm may each include an outer notch and an inner notch. The outer notch may be in communication with the inner notch. Also, the plurality of notches of the second elongate arm may each include an outer notch and an inner notch. The outer notch of the second proximal element may be in communication with the inner notch of the second proximal element.

Further, the first guard rail of the respective first and second proximal elements may define a plurality of elongate openings each being in communication with a respective one of the plurality of notches formed in the first side edge, and the second guard rail of the respective first and second proximal elements may define a plurality of elongate openings in communication with a respective one of the plurality of notches formed in the second side edge. The first and second guard rails of the respective first and second proximal elements may be connected to the respective first and second side edges at a plurality of locations.

Also, each frictional element of the first and second elongate arms may include a prong. The first and second elongate arms may each define a first width extending in a direction transverse to a longitudinal axis thereof, and the first and second guard rails of the first and second proximal elements may collectively define a second width greater than the first width. The frictional elements of the respective first and second elongate arms may be arranged in rows along a first length, and the first and second guard rails of respective first and second proximal elements may extend along a second length greater than the first length. The frictional elements of the respective first and second elongate arms may be arranged in rows, and the first and second guard rails of respective first and second proximal elements may extend beyond each of the rows of frictional elements in an axial direction. The first and second elongate arms may each extend from a first end portion to a second end portion. The frictional elements may each be disposed between the first and second end portions, and the first and second guard rails may be connected to the first and second end portions and extend therebetween.

In another aspect of the present disclosure, a fixation device may include a first distal element and a second distal element. The fixation device may also include a first proximal element disposed in opposition to the first distal element and moveable relative thereto. The first proximal element may have a plurality of frictional elements that may define a plurality of openings extending through the first proximal element at first and second sides thereof. The first proximal element may also having a first guard rail extending along the first side of the first proximal element and a second guard rail extending along the second side of the first proximal element. The fixation device may further include a second proximal element disposed in opposition to the second distal element and moveable relative thereto. The second proximal element may have a plurality of frictional elements that may define a plurality of openings extending through the second proximal element at first and second sides thereof. The second proximal element may also have a first guard rail extending along the first side of the second proximal element and a second guard rail extending along the second side of the second proximal element.

Additionally, the first and second distal elements may each have a first end portion, a second end portion, and a tissue engagement surface extending between the first and second end portions of the respective first and second distal elements. The first proximal element may include a first elongate arm that may have first and second side edges at least partially defining the first and second sides of the first proximal element. The first and second guard rails of the first proximal element may respectively flank the first and second side edges of the first elongate arm and at least partially define the first and second sides of the first proximal element. The second proximal element may include a second elongate arm that may have first and second side edges at least partially defining the first and second sides of the second proximal element. The first and second guard rails of the second proximal element may respectively flank the first and second side edges of the first elongate arm and at least partially define the first and second sides of the second proximal element.

Further, the first proximal element may further include inner rails and transverse rails. The inner rails, transverse rails, and first and second guard rails of the first proximal element may collectively define the openings of the first proximal element, and the frictional elements of the first proximal element may each extend from a corresponding transverse rail. The second proximal element may further include inner rails and transverse rails. The inner rails, transverse rails, and first and second guard rails of the second proximal element may collectively define the openings of the second proximal element, and the frictional elements of the second proximal element may each extend from a corresponding transverse rail. The first and second guard rails of each of the first and second proximal elements may each have a varying width.

Also, the frictional elements of the first and second proximal elements may be tilted inwardly toward a longitudinal axis of the respective first and second proximal elements. The first and second proximal elements may each include a fixed end and a free end. The free end may be bent in a direction away from the respective first and second distal elements and may including an opening configured to receive a proximal element line configured to actuate the respective first and second proximal elements between a first position and a second position.

In another aspect of the present disclosure, a fixation device may include a first distal element and a second distal element. The fixation device may also include a first proximal element and a second proximal element each disposed in opposition to the respective first and second distal elements and may each include an elongate arm and a first guard rail and a second guard rail. The elongate arm may have a plurality of frictional elements extending therefrom in a direction toward the respective first and second distal elements and arranged at intervals along a first length. The first and second guard rails may extend adjacent to the elongate arm along a second length greater than the first length.

In a further aspect of the present disclosure, a gripping device may include a base section, a first proximal element, and an arm bend feature. The first proximal element may have a first end portion, a second end portion, first and second side edges extending between the first and second end portions, and a plurality of frictional elements disposed between the first and second end portions. The first proximal element may also have a first guard rail extending along and adjacent to the first side edge of the first proximal element and a second guard rail extending along and adjacent to the second side edge of the first proximal element. The first bend feature may be coupled to the base section and the first end portion of the elongate arm. The first bend feature may be configured to flex to allow the first proximal element to move between first and second positions.

### BRIEF DESCRIPTION OF THE DRAWINGS

FIG. 1A is a cross-sectional representation of a heart illustrating its four valves.

FIG. 1B is a cross-sectional representation of a heart illustrating the left ventricle and left atrium during systole.

FIG. 2A is a schematic view of a mitral valve during normal coaptation.

FIG. 2B is a schematic view of a mitral valve during regurgitate coaptation.

FIGS. 3A and 3B are schematic views of a fixation device according to an embodiment of the present disclosure grasping leaflets of a mitral valve.

FIG. 4A is a perspective view of a fixation device according to another embodiment of the present disclosure.

FIG. 4B is a perspective view of the fixation device of FIG. 4A including a covering.

FIG. 5A is a perspective view of a gripping device of the of the fixation device of FIG. 4A according to an embodiment of the present disclosure.

FIG. 5B is an elevational view of the gripping device of FIG. 5A.

FIG. 6A is a perspective view of a gripping device according to another embodiment of the present disclosure.

FIG. 6B is a partial schematic view of the gripping device of FIG. 6A coupled to a distal element of the fixation device of FIG. 4A.

FIG. 6C is a partial schematic view of a gripping device according to an alternative embodiment of the present disclosure coupled to a distal element according to an alternative embodiment of the present disclosure.

FIG. 7A is an elevational view of a coupling system according to an embodiment of the present disclosure for coupling the fixation device of FIG. 4A and a delivery system.

FIGS. 7B and 7C are schematic views of the coupling system of FIG. 7A in respective first and second configurations.

FIGS. 8A and 8B are schematic cross-sectional views of a coupling system according to another embodiment of the present disclosure for coupling a fixation device, such as the fixation device of FIG. 4A, and a delivery system.

FIGS. 9A-9B, 10A-10B, 11A-11B, 12A-12B and 13A-13C illustrate the fixation device of FIG. 4A in various possible positions during introduction and placement of the device within a mammalian body to perform a therapeutic procedure.

FIG. 14 is a perspective view of the fixation device of FIG. 4A including a locking mechanism according to an embodiment of the present disclosure and illustrating a plurality of proximal element lines and a lock line coupled to the fixation device.

FIG. 15 is an elevational view of the locking mechanism and proximal elements of the fixation device of FIG. 14 and illustrating a lock line and single proximal element line respectively coupled thereto.

FIG. 16 is a schematic view of the fixation device of FIG. 4A coupled to a delivery system and illustrating a plurality of proximal element lines coupled to a shaft of the delivery system.

FIGS. 17A and 17B are partial enlarged views of a distal end portion of the delivery system shaft of FIG. 16 according to an embodiment of the present disclosure.

FIG. 17C is a cross-sectional view of the delivery system shaft taken along line C-C of FIG. 17B.

FIG. 17D is a partial perspective view of a distal end portion of one of the proximal element lines of FIG. 16 including a catch element according to an embodiment of the present disclosure.

FIG. 17E is a partial elevational view of the delivery system shaft of FIG. 17A having holes configured to receive the catch element of FIG. 17D.

FIG. 17F is a partial elevational view of the delivery system shaft of FIG. 17A and an actuator rod disposed therein intersecting the holes of the delivery system shaft.

FIG. 17G is a partial elevational view of a distal end portion of one of the proximal element lines of FIG. 16 including a catch element according to another embodiment of the present disclosure.

FIG. 18A is an enlarged cross-sectional view of the locking mechanism of FIG. 14 taken along a midline thereof and in an unlocked configuration.

FIG. 18B is an enlarged elevational view of the locking mechanism of FIG. 14 and in a locked configuration.

FIG. 18C is a perspective view of a release harness of the locking mechanism of FIG. 14.

FIG. 19A is an elevational view of a locking mechanism of the fixation device of FIG. 4A according to another embodiment of the present disclosure.

FIG. 19B is a transparent perspective view of a binding plate of the locking mechanism of FIG. 19A.

FIG. 19C is an enlarged elevational view of the locking mechanism of FIG. 19A.

FIG. 20 illustrates tissue becoming inadvertently snagged in a proximal element of the gripping device of FIG. 5A.

FIG. 21A is a top perspective view of a gripping device according to another embodiment of the present disclosure.

FIG. 21B is a side perspective view of the gripping device of FIG. 21A.

FIG. 21C is a bottom perspective view of the gripping device of FIG. 21A.

FIG. 21D is a top view of the gripping device of FIG. 21A.

FIG. 21E is a partial bottom view of the gripping device of FIG. 21A.

FIG. 21F is a partial bottom view of a gripping device according to a further embodiment of the present disclosure.

FIG. 22 is a schematic bottom view of a proximal element according to a further embodiment of the present disclosure.

FIG. 23 is a schematic bottom view of a proximal element according to another embodiment of the present disclosure.

FIG. 24A is schematic bottom view of a proximal element according to a further embodiment of the present disclosure.

FIG. 24B is a schematic end view of the proximal element of FIG. 24A.

FIG. 25 is a schematic side view of a proximal element according to a further embodiment of the present disclosure.

FIG. 26A is a partial perspective view of a gripping device according to another embodiment of the present disclosure.

FIG. 26B is a partial top view of the gripping device of FIG. 26A.

FIG. 26C is a partial end view of the gripping device of FIG. 26A.

FIG. 26D is a partial side view of the gripping device of FIG. 26A in an exemplary first configuration.

FIG. 26E is a partial side view of the gripping device of FIG. 26A in an exemplary second configuration

FIG. 27A is a partial top view of a gripping device according to a further embodiment of the present disclosure.

FIG. 27B is a partial bottom perspective view of the gripping device of FIG. 27A.

FIG. 27C is a partial top view of a gripping device according to another embodiment of the present disclosure.

FIG. 27D is a partial perspective view of the gripping device of FIG. 27C in an exemplary first configuration.

FIG. 27E is a partial perspective view of the gripping device of FIG. 27C in an exemplary second configuration and engaging tissue.

FIG. 27F is a partial top view of a gripping device according to a further embodiment of the present disclosure.

FIG. 28A is a partial perspective view of a gripping device according to another embodiment of the present disclosure.

FIG. 28B is a partial side view of the gripping device of FIG. 28A.

FIG. 28C is a perspective view of a modular guard rail of the gripping device of FIG. 28A.

FIG. 29A is a partial perspective view of a gripping device according to a further embodiment of the present disclosure in an exemplary first configuration.

FIG. 29B is a partial top view of the gripping device of FIG. 29A in an exemplary second configuration and engaging tissue.

FIG. 29C is a partial side view of the gripping device of FIG. 29A in the exemplary second configuration and engaging the tissue.

FIG. 29D is a partial perspective view of the gripping device of FIG. 29A in the exemplary second configuration and engaging the tissue representative tissue.

FIG. 29E is a partial perspective view of a gripping device according to another embodiment of the present disclosure.

FIG. 29F is a partial perspective view of a gripping device according to a further embodiment of the present disclosure.

### DETAILED DESCRIPTION

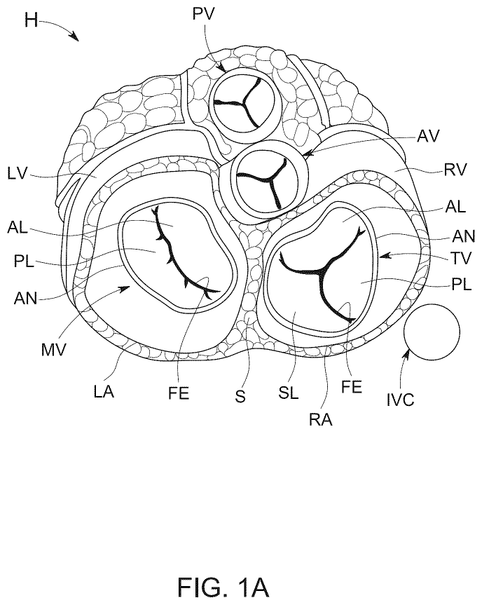

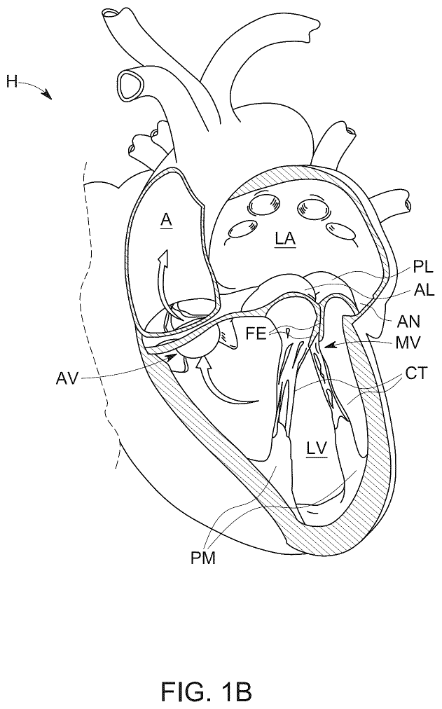

The valves of a normal heart H are illustrated in FIGS. 1A and 1B. These valves include the mitral valve MV, the tricuspid valve TV, the aortic valve AV, and the pulmonary valve PV. The mitral valve MV separates the left atrium LA and the left ventricle LV, and the tricuspid valve TV separates the right atrium RA and the right ventricle RV. The mitral valve MV and the tricuspid valve TV are sometimes referred to as the atrioventricular valves. The mitral valve MV is a bicuspid valve in that it has two leaflets referred to as the posterior leaflet PL and the anterior leaflet AL. The tricuspid valve TV typically has three leaflets referred to as the anterior leaflet AL, the posterior leaflet PL, and the septal leaflet SL. However, studies have shown that, although the TV is typically composed of three leaflets of unequal size, in many cases, two or more than three leaflets may be present as anatomic variants in healthy subjects. Thus, reference herein to the tricuspid valve TV should be understood to refer to the atrioventricular valve located between the right atrium RA and right ventricle RV regardless of the number of leaflets be it two, three, or more than three leaflets. However, exemplary embodiments discussed herein refer to the usual anatomic structure of the tricuspid valve TV that includes three leaflets.

As illustrated in FIG. 1B, the anterior leaflet AL and posterior leaflet PL of the mitral valve MV extend from a valve annulus AN to respective free edges FE. The free edges FE are secured to the lower portions of the left ventricle LV through chordae tendineae CT (referred to hereinafter as the chordae). The chordae CT include a plurality of branching tendons that are attached to papillary muscles PM at the lower portions of the left ventricle LV and extend upwardly to the lower surfaces of each of the valve leaflets where they are attached. The three leaflets of the tricuspid valve TV similarly extend from a valve annulus AN to respective free edges FE which are secured via chordae to the papillary muscles of the right ventricle RV.

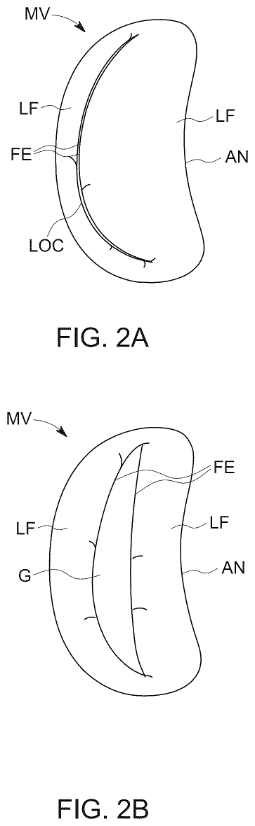

The mitral valve MV depicted in FIGS. 1B and 2A illustrate the proper functioning of an atrioventricular valve during ventricular systole. As the ventricles contract, the free edges FE of adjacent leaflets LF meet along a line of coaptation LOC. The joinder of the leaflets LF at this line of coaptation LOC seals off the ventricle from the atrium and prevents the back flow of blood or “regurgitation” from entering into the atrium. Thus, with the right atrium RA and left atrium LA respectively sealed off by the mitral valve MV and tricuspid valve TV, blood in the left ventricle LV can only flow through the aortic valve AV to the body, and blood in the right ventricle RV can only flow through the pulmonary valve PV to the lungs.

A number of structural defects in the heart H can cause mitral valve regurgitation (“MVR”) and/or tricuspid valve regurgitation (“TVR”). MVR and TVR occur when their respective leaflets LF do not close properly allowing leakage from the ventricle into the atrium. The mitral valve MV depicted in FIG. 2B illustrates valvular insufficiency of an atrioventricular valve resulting in regurgitation. In the depicted example, an enlargement of the heart H may cause the valve annulus AN to become enlarged, making it impossible for the free edges FE of the valve leaflets LF to meet during systole. This may result in a gap G between the leaflets LF which allows blood to leak through the valve. In another example, ruptured or elongated chordae CT can cause a valve leaflet LF to prolapse at least due to inadequate tension transmitted to the leaflet via the chordae CT. While an adjacent leaflet LF may maintain a normal profile, the prolapsing leaflets LF may flail about preventing the proper joinder between the leaflets LF resulting in leakage into the atrium. In a further example, regurgitation can occur in patients who have suffered ischemic heart disease which may result in weak ventricular contractions insufficient to effect proper closure.

The present disclosure describes exemplary systems, devices, and methods for percutaneously repairing a valve to treat cardiac valve regurgitation, particularly MVR and TVR. When referring to such disclosed systems, devices, and methods, the term “proximal” (P) shall mean closer to the user or in a direction toward a device to be manipulated by the user outside the patient's body, and the term “distal” (D) shall mean more distant from the user or in a direction toward a device that is positioned at the treatment site within the patient's body (e.g., fixation device 112). With respect to the mitral valve and tricuspid valve, “proximal” shall refer to the atrial or upstream side of the valve leaflets, and “distal” shall refer to the ventricular or downstream side of the valve leaflets.

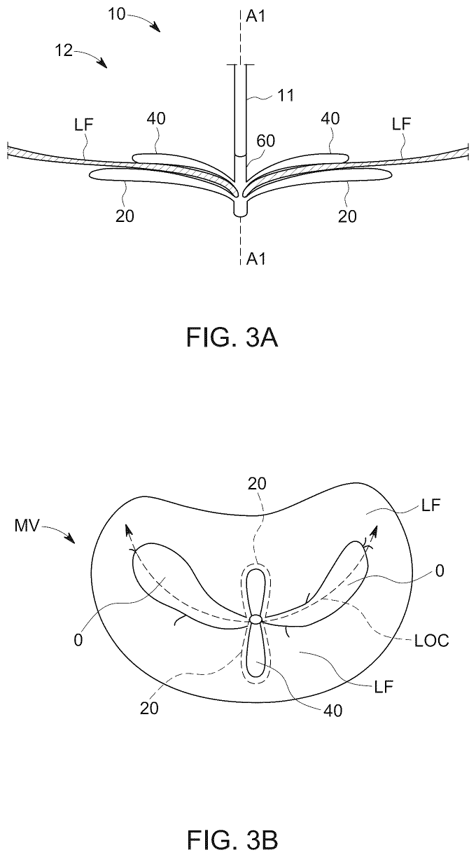

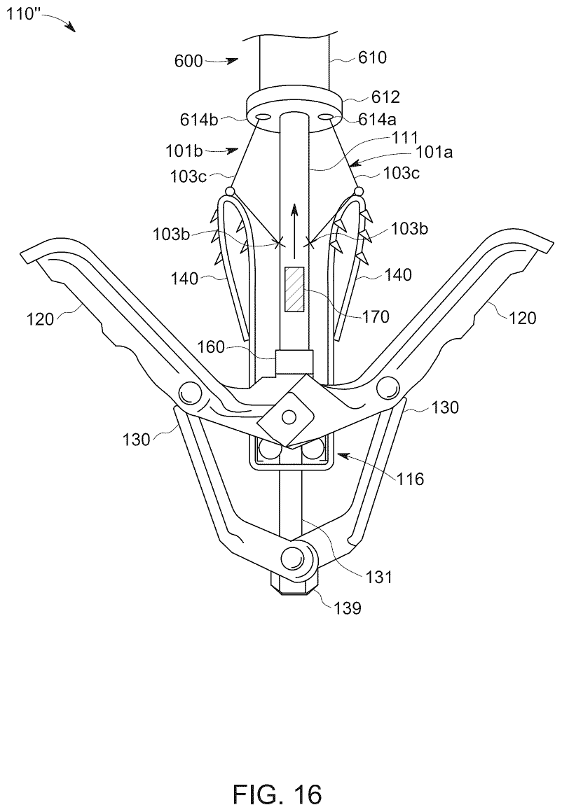

FIGS. 3A and 3B depict a fixation device 12, according to an embodiment of the present disclosure, grasping leaflets LF of an atrioventricular valve, which is illustrated as a mitral valve MV. Fixation device 12 may be releasably coupled to a distal end of a shaft 11 of a delivery system 600 (see FIG. 16) to form an interventional tool 10. Fixation device 12 may include distal elements 20 (also referred to herein as fixation elements) and proximal elements 40 (also referred to herein as gripping elements). Distal and proximal elements 20, 40 may be moveable relative to each other and may protrude radially outward relative to a longitudinal axis A1 of fixation device 12. As shown in FIG. 3A, fixation device 12 may be positionable on opposite sides of adjacent leaflets LF of the valve so as to capture or retain the leaflets LF therebetween. In this regard, proximal elements 40 may be positioned at a proximal side of the valve leaflets LF, and distal elements 20 may be positioned on a distal side of the valve leaflets LF. Proximal elements 40 may be made from cobalt chromium, nitinol, or stainless steel, for example, and distal elements 20 may be made from cobalt chromium or stainless steel, for example.

Fixation device 12 may be releasably coupled to shaft 11 such that it can be detached and left behind as an implant to hold the leaflets LF together in the coapted position. In this regard, fixation device 12 may be delivered to a target valve percutaneously using any one of a number of different approaches, such as via a transfemoral, a transapical, or a transjugular approach, for example. Thus, in one example of treating MVR, fixation device 12 may be delivered to the deficient mitral valve MV using a transfemoral approach in which fixation device 12 is guided through the inferior vena cava IVC (see FIG. 1A), across the interatrial septum S, and into left atrium LA where fixation device 12 is advanced into the mitral valve MV. Also, in one example of treating TVR, fixation device 12 may be guided transfemorally through the inferior vena cava IVC to the right atrium RA where fixation device 12 is advanced to a desired position within the tricuspid valve TV.

FIG. 3B is an atrial-side view of fixation device 12 in one example of a desired orientation in relation to adjacent leaflets LF of an atrioventricular valve, such as the depicted mitral valve MV. The distal and proximal elements 20, 40 are positioned to be substantially perpendicular to the line of coaptation LOC. Thus, in the case of a mitral valve MV, fixation device 12 may be oriented perpendicular (+/−5 degrees) to a line of coaptation LOC between the posterior leaflet PL and anterior leaflet AL, and in the case of a tricuspid valve TV, fixation device 12 may be positioned perpendicular (+/−5 degrees) to a line of coaptation between the septal leaflet SL and the anterior leaflet AL, the septal leaflet SL and the posterior leaflet PL, or the anterior leaflet AL and the posterior leaflet PL, for example. Device 12 may be moved roughly along the line of coaptation LOC to the location of regurgitation. The leaflets LF may be held in place so that, during diastole, the leaflets LF remain in position between elements 20, 40 surrounded by openings O (also referred to herein as orifices) which result from the diastolic pressure gradient. Advantageously, leaflets LF are coapted such that their proximal or upstream surfaces face each other in a vertical orientation, parallel to the direction of blood flow through the valve. The upstream surfaces may be brought together so as to be in contact with one another or may be held slightly apart but will preferably be maintained in the vertical orientation in which the upstream surfaces face each other at the point of coaptation. This simulates the double orifice geometry of a standard surgical bow-tie repair. Color Doppler echo will show if the regurgitation of the valve has been reduced. If the resulting flow pattern is satisfactory, the leaflets LF may be fixed together in this orientation. If the resulting color Doppler image shows insufficient improvement in valve regurgitation, fixation device 112 may be repositioned. This may be repeated until an optimal result is produced wherein the leaflets LF are held in place.

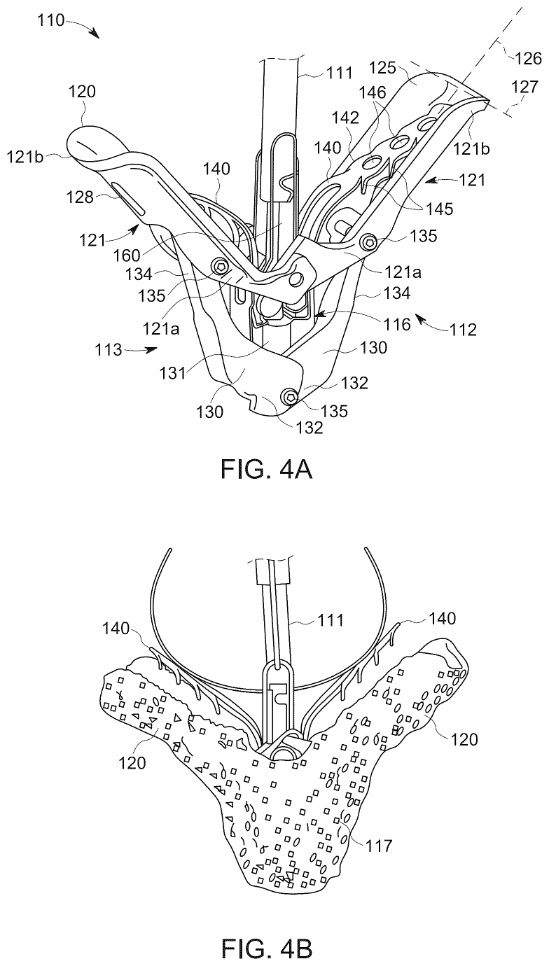

FIGS. 4A-19C depict a fixation device 112 according to another embodiment of the present disclosure. Fixation device 112 may generally include a pair of distal elements 120, a pair of proximal elements 140, a coupling member 160, an actuator 113, and a stud 131. Distal elements 120 may include elongate arms 121 in which each arm has a proximal end portion 121a, which may be rotatably connected to the coupling member 160, and a free end 121b, as best shown in FIG. 4A. Free ends 121b may each have a rounded shape to minimize interference with and trauma to surrounding tissue structures according to one example. In one example, each free end 121b defines a curvature extending about two axes 126, 127. The first axis 126 may be a longitudinal axis of each respective arm 121. Additionally, arms 121 may each include an engagement surface 125 that may also be curved about first axis 126 and may extend at least partially along a length of arm 121 to the free end 121b. Thus, in some examples, engagement surfaces 125 may each have a cupped or concave shape which may maximize contact area engagement with tissue and may assist in grasping and holding valve leaflets. Such cupped or concave shape may further allow arms 121 to nest around shaft 111 of interventional tool 110 while in the closed position to minimize the profile of device 112. Thus, arms 121 may be at least partially cupped or curved inwardly about their longitudinal axes 126 which may form a concavity extending along axis 126 which may nest proximal elements 140 when in a lowered position thereof. The second axis 127 about which each free end 121b may be curved may extend perpendicular to first axis 126, as is also shown in FIG. 4A. The curvature about this second axis 127 may be a reverse curvature located at the most distal portion of free ends 121b. In addition to the dual curvature, free ends 121b may flare outwardly at their respective longitudinal edges. It is believed that both the reverse curvature and flare help create an atraumatic configuration that minimizes trauma to the tissue engaged therewith.

In the nonlimiting embodiment depicted, a transverse width across engagement surfaces 125 (which is in the direction of second axis 127 and determines the width of tissue engaged) may be at least about 2 mm, 3-10 mm in some examples, and about 4-6 mm in some examples. In some embodiments, a wider engagement may be desired wherein the engagement surfaces 125 are larger, for example about 2 cm, or multiple fixation devices 112 may be used adjacent to each other. Arms 121 may also have a length of about 6-12 mm (defined along first axis 126), and engagement surfaces 125 may be configured to engage a length of tissue of about 4-10 mm along the longitudinal axis 126 of arms 121 according to some examples. Also, as shown in the illustrated example, each arm 121 may include a plurality of openings 128 to enhance grip and to promote tissue ingrowth following implantation.

In one example, actuator 113 may include two link members or legs 130. Legs 130 may be comprised of a rigid or semi-rigid metal or polymer such as Elgiloy®, cobalt chromium or stainless steel, however any suitable material may be used. Each leg 130 may have a first end 132, which may be rotatably joined with one of the distal elements 120 at a riveted joint 135, and a second end 134, which may be rotatably joined with stud 131, as shown in FIG. 4A. Although the depicted embodiment shows both legs 130 pinned to stud 131 by a single rivet 135, it is also contemplated that each leg 130 may be individually attached to the stud 131 by a separate rivet, pin or the like. In other embodiments of actuator 113, actuator 113 may include a base 139, and second ends 134 of legs 130 may be rotatably joined with base 169, such as by one or more riveted joints 135, as best shown in FIG. 10B. An actuator rod 170 of delivery 600 may be joinable with actuator 113 directly, such as via direct connection with base 139, or indirectly, such as via connection with stud 131, which itself may extend from base 139. In either of these embodiments, actuator rod 170 may be axially extendable and retractable in a proximal-distal direction to actuate actuator 113 and consequently rotate distal elements 120 between open, closed, and inverted positions, which are described further below. Additionally, coupling member 160, stud 131, and/or base 169 may comprise a center portion or center body of fixation device, for example.

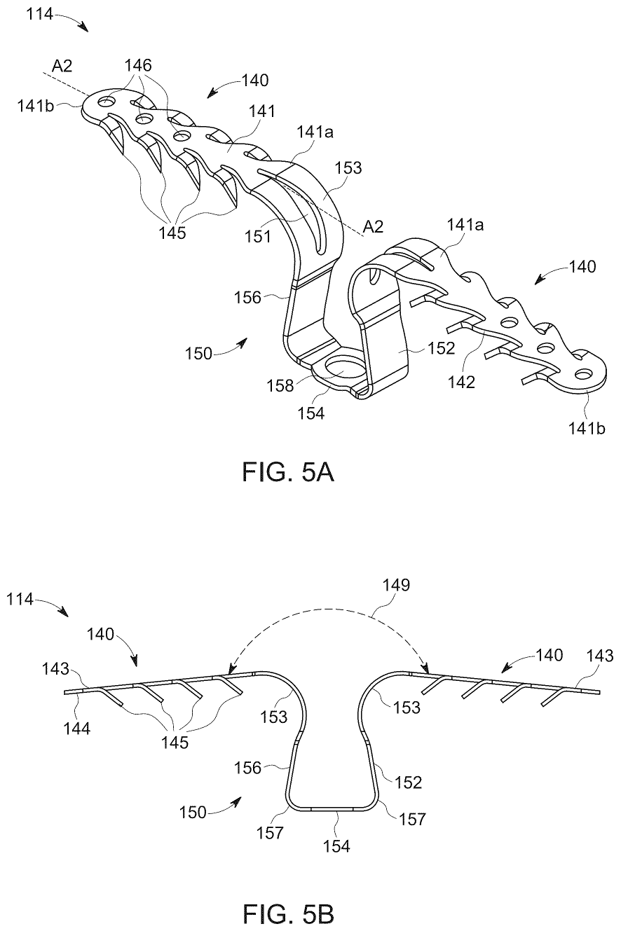

Proximal elements 140 may, in some examples, be flexible, resilient, and cantilevered from a center of fixation device 112. For example, FIGS. 5A and 5B depict a gripping device 114 according to an embodiment of the present disclosure that may generally include a pair of proximal elements 140, a base section 150, and a pair of arm bend features 153 partitioning proximal elements 140 from base section 150.

Proximal elements 140 may be in the form of elongate arms 141 that each extend along a longitudinal axis A2 from a first end portion or fixed end 141a to a second end portion or free end 141b, as shown in FIG. 5A. Each proximal element 140 may also have opposed side edges 142 that define a width transverse to the longitudinal axis A2. Such width may be less than the width of a corresponding distal element 120 such that proximal element 140 may be recessed within the concavity formed by engagement surface 125 of distal element 120 when proximal element 140 is moved into a lowered position, as described in more detail below.

Proximal elements 140 may also each have a first side or proximal side 143 and a second side or distal side 144. In one example, proximal elements 140 may include a plurality of openings 146 that may extend from proximal side 143 to distal side 144, as shown in FIG. 5A. Such openings 146 may be used to couple a proximal element line, which is discussed further below, to a proximal element 140 for raising and lowering proximal element 140. Each proximal element 140 may also include one or more frictional elements 145 extending from distal side 144. For example, each proximal element 140 may include one or more rows of frictional elements 145 where frictional elements 145 in each row may be aligned in a direction transverse to longitudinal axis A2. Frictional elements 145 in such rows may also be aligned with frictional elements 145 in other rows in a lengthwise direction thereby forming columns of frictional elements 145. For example, in the embodiment depicted in FIGS. 5A and 5B, each proximal element 145 may include four rows of two frictional elements 145. In other words, two columns of four frictional elements 145. In other embodiments, proximal elements 140 may include one to six rows of two to six frictional elements 145 per row, for example. However, in other embodiments, frictional elements 145 may be arranged in an offset relationship in a lengthwise and/or transverse direction such that at least some frictional elements 145 are not aligned with another frictional element 145 in such directions.

Frictional elements 145 may comprise frictional protrusions or tines having tapering pointed tips extending from distal side 144 of proximal elements 140. Frictional elements 145 may also be angled toward fixed end 141a of proximal element 140 which may help prevent frictional elements 145 from inadvertently snaring tissue during repositioning of fixation device 112. In one example, frictional elements 145 may be integral with or connected to a distal surface 144 of a proximal element 140 and protrude therefrom. In another example, as shown in FIG. 5A, frictional elements 145 may be formed from side edges 142, such as by cutting and bending the base material forming proximal elements 140, for example. It may be appreciated that any suitable frictional elements may be used, such as prongs, windings, bands, barbs, grooves, channels, bumps, surface roughening, sintering, high-friction pads, coverings, coatings, or a combination of these. However, it should be noted that some types of frictional elements that can be utilized may permanently alter or cause some trauma to the tissue engaged. Thus, it is preferable that frictional elements 145 be atraumatic and generally frictional rather than penetrative so as to not injure or otherwise affect the tissue in a clinically significant way.

Base section 150 may be connected to a center portion or center body of fixation device 112 such that proximal elements 140 extend outwardly therefrom. For example, base section 150 may be coupled to coupling member 160. In the embodiment depicted, base section 150 may include a first member 152, a second member 154, and a third member 156. First and third members 152, 156 may be connected to second member 154 to form a generally U-shaped or box-shaped structure which may allow a lock (discussed below) to be positioned between first and third members 152, 156. However, other shapes may be formed, such as a V-shape, a crescent shape, or semicircular, for example. In some embodiments, first and third members 152, 156 may be connected to second member 154 via base bend features 157, for example. Also, second member 154 may include an opening 158 extending therethrough for receipt of stud 131 and/or actuator rod 170, as shown in FIG. 5A.

Arm bend features 153 may couple a respective proximal element 140 and base section 150. For example, an arm bend feature 153 can couple a proximal element 140 to first member 152 of base section 150, and another arm bend feature 153 can coupled the other proximal element 140 to third member 156 of base section 150. As shown, arm bend features 153 may form a living hinge about which proximal elements 140 may bend relative to base section 150. In this regard, arm bend features 153 may be integral with proximal elements 140 and base section 150 and may bias proximal elements 140 to a relaxed position. As illustrated in FIG. 5B, proximal elements 140 may form a relaxed angle 149 formed between proximal sides 143 of each proximal element 140. Such relaxed angle 149 is formed when proximal elements 140 are in the relaxed position and may form an angle of about 85 degrees to 200 degrees (+/−5 degrees). For example, proximal elements 140 may form a relaxed angle of 180 degrees in the relaxed position. In another example, proximal elements 140 may form a relaxed angle of 185 degrees in the relaxed position. Although the embodiment depicted illustrates bend features 153 as living hinges, in other embodiments bend features 153 may comprise a biased hinge that modularly connects proximal elements 140 to base section 150. For example, proximal elements 140 may be separately formed from base section 150 and modularly connected to base section 150 via arm bend features 153 which may each comprise a spring biased hinge biasing a respective proximal element 140 to the relaxed position, for example.

Arm bend features 153 may also each include an elongate opening extending 151 along the longitudinal axis A2 which may furcate each arm bend feature 153, as illustrated in FIG. 5A. Such an elongated opening 151 may have a uniform width extending along axis A2. However, in some embodiments, such as the embodiment depicted, elongate opening 151 may form a bowling-pin shape such that a width of opening 151 is narrower at one end (e.g., the end closest to free end 141b) than the other end (e.g., the end furthers from free end 141b) and is wider somewhere in between. Elongate opening 151 may also not be relegated to just arm bend feature 153 but may also extend from arm bend feature 153 to proximal element 140 and/or base body 150. The elongate opening 151 and corresponding furcation of arm bend features 153 may be configured (e.g., in size, shape, spacing, position, etc.) so as to provide the desired resiliency, fatigue resistance, and/or flexibility at the coinciding arm bend features 153.

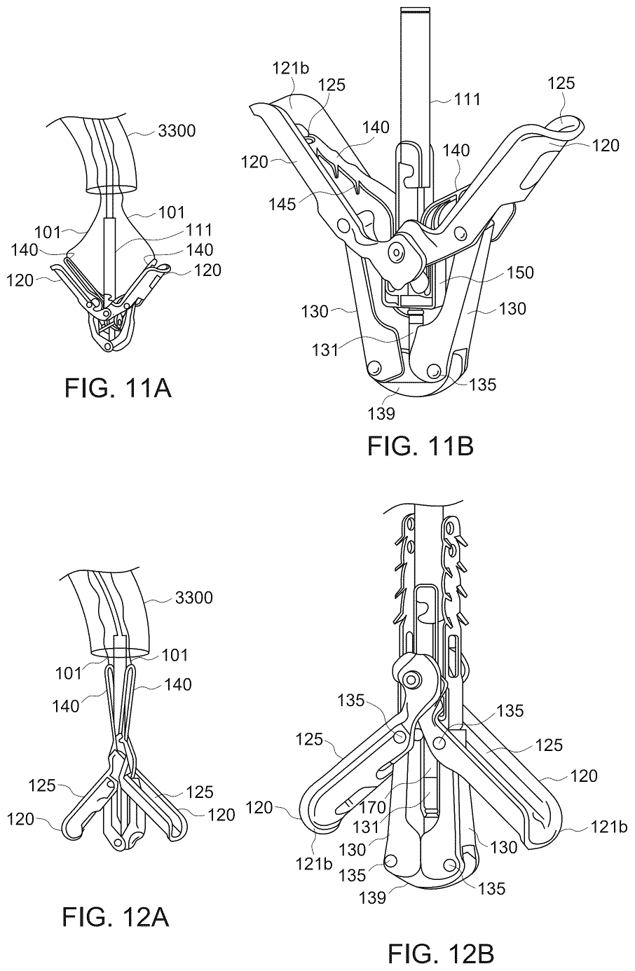

Base bend features 110 and arm bend features 112 may be configured to give gripping device 116 a bent configuration when gripping device is in a relaxed state (i.e., when proximal elements are in the relaxed position), such that when gripping device 114 is forced into a stressed state (e.g., by bending proximal elements at one or more of the base and/or arm In the exemplary embodiment depicted, gripping device 114 may be formed from a metallic sheet of a spring-like material, such as a shape-memory metal (e.g., Nitinol) which may provide the bias of proximal elements 140 toward the relaxed position. Alternatively, gripping device 114 could be molded from a biocompatible polymer. Each proximal element 112 may, in one example, be configured to be at least partially recessed within the concavity of the distal element 120 when no tissue is present. When fixation device 112 is in the open position, each proximal element 140 may be separated from the engagement surface 125 near free end 121b of arm 121 and may slope toward engagement surface 125 near free end 121b with the free end 141b of proximal element 140 contacting engagement surface 125, as illustrated in FIGS. 4A and 11B. This arrangement may be facilitated by the dimensions of base section 150. For example, increasing or decreasing the respective lengths of first, second, and third members 152, 154, 156 of base section 150 may increase or decrease the separation distance between a proximal element 140 and corresponding distal element 120 which may help accommodate a valve leaflet or other tissues of varying thicknesses. Further examples of gripping devices that may be utilized in fixation device 112 are described in more detail in U.S. Pat. No. 11,096,691, the disclosure of which is incorporated by reference herein in its entirety.

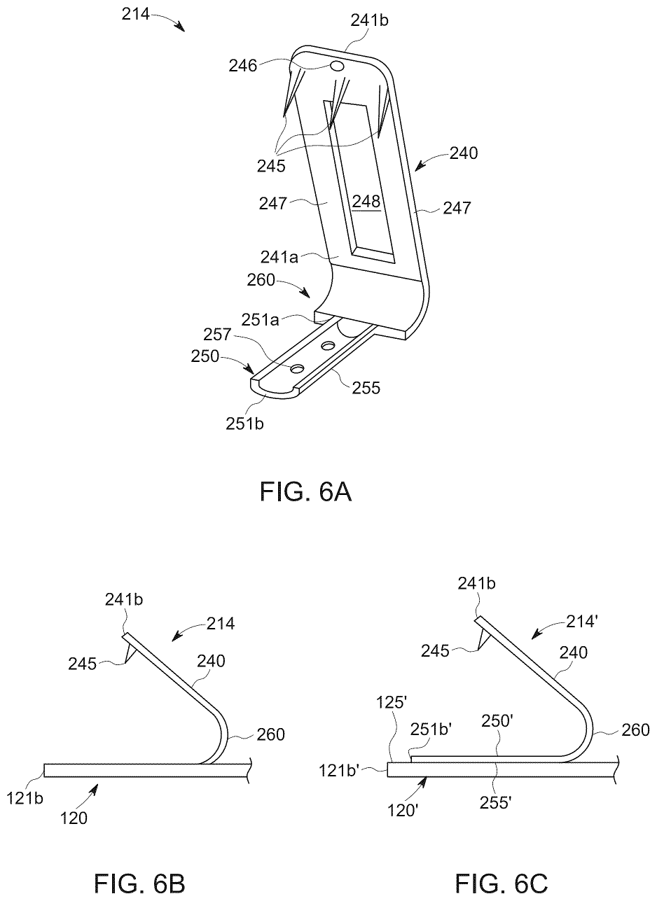

In other embodiments proximal elements may be connected to or otherwise extend from distal elements rather than from a center of fixation device, like that of fixation device 112. For example, FIGS. 6A and 6B depict a gripping device 214 according to another embodiment of the present disclosure that may generally include a first arm 240, a second arm 250, and an arm bend feature 260 partitioning first arm 240 from second arm 250. Gripping device 214 may be made from a shape-memory-metal material, such as Nitinol, for example.

First arm 240 may constitute a proximal element of fixation device 112, like that of and as an alternative to proximal element 140 and may include one or more frictional elements 245 which may be similar to frictional elements 145 discussed above. Thus, a plurality of frictional elements 245 may extend from a distal side of first arm 240 such as in one or more rows and/or columns. In the embodiment depicted, a single row of three frictional elements 245 may be provided near a free end 241b of first arm 240. But, as mentioned above, first arm 240 may have any number of frictional elements 245, such as two, four, or six, for example. First arm 240 may also include a pair of elongate members 247 offset from each other to form a space 248 therebetween. Such space 248 may be configured to receive second arm 250, for example. Additionally, first arm 240 may include one or more openings 246, such as near free end 241b, as shown in FIG. 6A. Such opening 246 may be configured to receive a proximal element line for raising and lowering first arm 240.

Second arm 250 may be in the form of a beam or other elongate structure. Second arm 250 (also referred to herein as base section) may be configured to couple to a distal element 120. For example, in the embodiment depicted in FIGS. 6A and 6B, second arm 250 may be curved in a plane transverse to its longitudinal axis. For example, second arm 250 may be semi-cylindrical such that it may have a semi-circular profile. Thus, second arm 250 may have a convex surface 255 configured to conform to the cupped curvature of engagement surface 125 of a corresponding distal element 120. FIG. 6B illustrates second arm 250 coupled to proximal engagement surface 125 of distal element 120 such that it is generally recessed within distal element 120 and free ends 241b, 251b of first and second arms 240, 250 point in the general direction toward free end 121b of distal element 120. Thus, in some embodiments, second arm 250 may have a width configured to be positioned within the concavity of distal element 120 and secure to proximal engagement surface 125. In other embodiments, a second arm 250′ of an alternative gripping device 214′ may not be concave and may instead have a planar surface corresponding to a planar engagement surface 125′ of an alternative distal element 120′ and secured thereto, as illustrated in FIG. 6C. In further embodiments, distal element 120 may include a recess or pocket for receipt and securement of second arm 250, such as in a press-fit manner, for example. Second arm 250 may be secured to distal element 120 in any number of ways, such as via one or more sutures, welding, press-fit, fastener (e.g., rivet or screw) or the like. For example, a rivet, screw, or suture may pass through one or more openings 257 in second arm 250 and into distal element 120. A tissue fixation device, such tissue fixation device 112, may include a pair of gripping devices 214 with one coupled to each distal element 120 as mentioned above.

Arm bend feature 260 may be coupled to a fixed end 241 of first arm 240 and a fixed end 251a of second arm 250 such that first and second arms 240, 250 extend in the same general direction and may form a V-shape when first arm 240 is in an exemplary open or raised position, as illustrated in FIGS. 6B and 6C. As shown, arm bend feature 260 may form a living hinge about which first arm 240 may bend relative to second arm 250. In this regard, arm bend feature 260 may be integral with first arm 240 and second arm 250 so as to form a monolithic structure and may bias first arm 240 to a relaxed position. Such relaxed position may include second arm 250 extending through space 248 between elongate members 247 of first arm 240 to form an X-shape. However, it should be noted that such position can generally only be achieved when gripping device 214 is not coupled to distal element 120 as the presence of distal element 120 would prevent second arm 250 from passing into space 248. It should also be appreciated that in some embodiments of gripping device 214, arm bend feature 260 may be a spring loaded or otherwise biased hinge coupling separately formed first and second arms 240, 250.

Fixation device 114 may also have a covering 117, as shown in FIG. 4B. As depicted, covering 117 may encapsulate distal elements 120 and actuator 113. Thus, engagement surfaces 125 may be covered by covering 117 which may help minimize trauma on tissues and enhance primary fixation via additional friction to assist in grasping. Additionally, covering 117 on engagement surfaces 125 may facilitate tissue ingrowth to provide for secondary fixation to ensure long-term security. Covering 117 may be loosely fitted and/or may be flexible such that device 112 can freely move to various positions all the while covering 117 conforms to the contours of the device 112 and remains securely attached thereto. It may be appreciated that the covering 117 may cover specific parts of fixation device 112 while leaving other parts exposed. For example, proximal elements 140 may be exposed, while distal elements 120 and actuator 113 may be covered. However, in some embodiments, proximal elements 140 may be covered with covering 117 to enhance grip and tissue ingrowth following implantation. Preferably, when a covering 117 is used in combination with frictional elements 145 or other frictional features, such as those extending from proximal elements 140, such features may protrude through such covering 117 so as to contact any tissue engaged by proximal elements 140.

Covering 117 may be comprised of any biocompatible material, such as polyethylene terepthalate, polyester, cotton, polyurethane, expanded polytetrafluoroethylene (ePTFE), silicon, or various polymers or fibers and have any suitable form, such as a fabric (woven or unwoven), mesh, textured weave, felt, looped or porous structure. Generally, covering 117 has a low profile so as not to interfere with delivery through an introducer sheath or with grasping and coapting of leaflets or tissue. Covering 117 may alternatively be comprised of a polymer or other suitable materials dipped, sprayed, coated, or otherwise adhered to the surfaces of the fixation device 112. Optionally, a polymer coating may include pores or contours to assist in grasping the tissue and/or to promote tissue ingrowth. Any of the coverings 117 may optionally include drugs, antibiotics, anti-thrombosis agents, or anti-platelet agents such as heparin, COUMADIN® (Warfarin Sodium), to name a few. These agents may, for example, be impregnated in or coated on the coverings 117. These agents may then be delivered to the grasped tissues surrounding tissues and/or bloodstream for therapeutic effects.

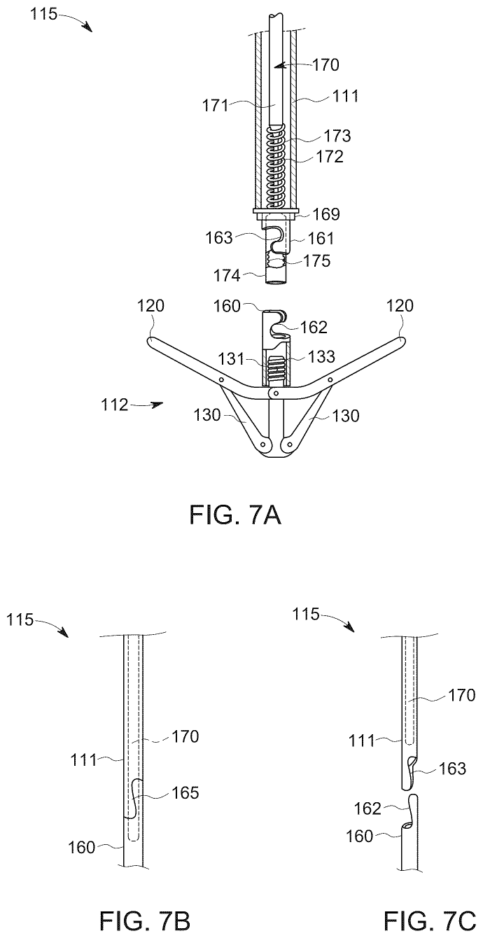

FIGS. 7A-7C depict an exemplary coupling system 115 between fixation device 112 and delivery system shaft 111. As mentioned above, once the leaflets of a target valve are coapted in the desired arrangement, fixation device 112 may then be detached from delivery 600 and left behind as an implant to hold the leaflets together in the coapted position. Such detachment may occur between coupling member 160 of fixation device 112 and a distal end of delivery shaft 111. Thus, coupling member 160 may be configured to be releasably coupled to shaft 111. Coupling member 160 may be disposed at a center of fixation device 112 and may extend proximally along it's the longitudinal axis of fixation device 112. In the coupling system 115 depicted, shaft 111 may form a tubular upper shaft with a first mating surface 163 formed at a distal end thereof, and coupling member 160 may form a detachable lower tubular shaft with a second mating surface 162 formed at a proximal end thereof. Mating surfaces 162, 163 may be correspondingly shaped so that they interlock and form a joining line 165 when merged together, as shown in FIG. 7B. In this regard, mating surfaces 162, 163 may have any shape or curvature which allows or facilitates interlocking and later detachment. For example, in the depicted embodiment, mating surfaces 162, 163 define a joining line 165 with an S-shaped curvature.

Coupling system 115 may also include actuator rod 170 and stud 131 (or alternatively base 139) such that fixation device 112 may also be releasably coupled to delivery 600 via connection between actuator rod 170 and stud 131. When shaft 111 is coupled to coupling member 160, they may collectively form an axial channel. Actuator rod 170 may pass through this channel to bridge the joining line 165, as shown in FIG. 7B. Actuator rod 170 may comprise a proximal extremity 171, a distal extremity 172, and a joiner 174. Distal extremity 172 may be smaller in diameter than proximal extremity 171 and may be optionally surrounded by a coil 173 which may serve to bias joiner 174 in a proximal direction. However, in some embodiments, actuator rod 170 may not have coil 173 or proximal and distal extremities 171, 172 of differing diameters. Joiner 174 may be removably coupled with stud 131 of fixation device 112 via any one of various possible release mechanisms. For example, in the embodiment depicted, joiner 174 may be threadedly connected to stud 131 of fixation device 112. In this regard, joiner 174 may have internal threads 175 which mate with external threads 133 on stud 131. Alternatively, joiner 174 may have external threads which mate with internal threads of stud 131. As described previously, stud 131 may be connected with distal elements 120 so that advancement and retraction of stud 131, by means of actuator rod 170, manipulates distal elements 120. It is also contemplated that joiner 174 may be directly threadedly engaged with base 139 where no stud 131 is provided. Once detachment of fixation device 112 is desired, actuator rod 170 may be rotated until threads 175 of joiner 174 disengage threads 133 of stud 131. Actuator rod 170 may then be retracted to a position above mating surfaces 162, 163 which in turn allows coupling member 160 to separate from shaft 111 along joining line 165, as illustrated in FIG. 7C.

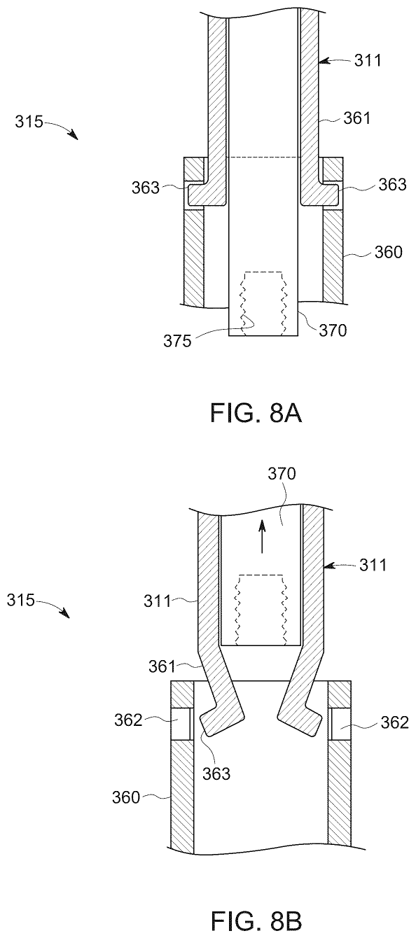

FIGS. 8A and 8B illustrate an alternative example of a coupling system. In this exemplary coupling system 315, shaft 311 of the delivery system (e.g., delivery 600) may be releasably coupled with coupling member 360 via a detent mechanism, for example. In this regard, shaft 311 may form an upper tubular shaft with detent mechanism features and coupling member 360 may form a lower tubular shaft with detent mechanism features configured to releasably connect with the detent mechanism features of shaft 311. In the embodiment depicted, the detent mechanism may include one or more spring arms 361 integrally formed on shaft 311 and one or more receptacles 362 sized to receive spring arms 361 within coupling member 360. However, shaft 311 may include receptacles 362, while coupling member 360 may include spring arms 361, for example. As shown, spring arms 361 may have a flange-like engagement element 363 at a distal end thereof and are preferably biased inwardly, i.e., toward an interior shaft 311, as shown in FIG. 8B. Receptacles or apertures 362 may be configured to receive and mate with respective engagement elements 363 of spring arms 361, as shown in FIG. 8A. Receptacles 362 may extend all the way through the wall of coupling member 360 and may be sized to snuggly fit both engagement elements 362. A snuggly fitting rod (such as actuator rod 370) may extend through shaft 311 and coupling member 360 and may outwardly deflecting the inwardly biased spring arm(s) 361 such that the engagement elements 363 are pushed into respective engagement with a corresponding receptacle 362 thereby coupling the shaft 311 to coupling member 360, as shown in the example of FIG. 8A. When desirable to detach fixation device 112 from delivery 600, actuator rod 370 may be retracted to a position above spring arm(s) 361 and engagement features 363 thereof. This allows the inwardly biased spring arms 361 and corresponding engagement elements 363 to disengage from receptacles 362 thereby detaching shaft 311 and coupling member 360. As mentioned above, actuator rod 370 may be threadedly engaged to stud 131. Thus, actuator rod 370 may first be rotated to unthread its threads 375 from stud 131 and then retracted to release coupling member 360 according to an example of the disclosure.

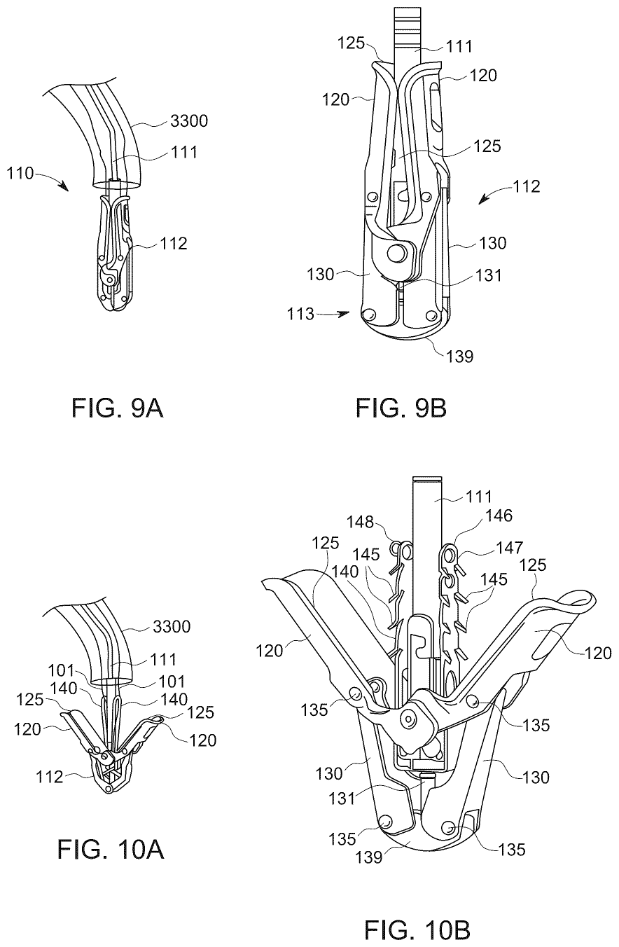

As mentioned above, fixation device 112 may, in one example, be actuated through multiple positions within a mammalian body during a transcatheter procedure such as by extending and retracting actuator rod 170 when coupled to stud 131 and/or base 139. FIGS. 9A-9B, 10A-10B, 11A-11B, 12A-12B, and FIGS. 13A-13B illustrate several of these possible positions and in a sequence that may be utilized during a transcatheter, therapeutic procedure (e.g., tissue approximation).

FIGS. 9A and 9B depict fixation device 112 in an example of a closed position or delivery position. Fixation device 112 may assume the closed position when being delivered through a guide catheter or sheath 3300 of a steerable guide system. In the closed position, the opposed pair of distal elements 120 may be positioned so that engagement surfaces 125 thereof face each other. The cupped or concave shape of each arm 121 in this example allows arms 121 to surround shaft 111 and optionally contact each other on opposite sides of shaft 111. This provides a low profile for fixation device 112 so that it is readily passable through a delivery catheter 3300 and through any anatomical structures, such as those within the cardiovascular system.

FIGS. 10A-10B depict fixation device 112 in an example of an open position. Fixation device 112 may assume the open position for capturing and grasping leaflets of a heart valve. In an open position, distal elements 120 may be rotated so that engagement surfaces 125 thereof face a first direction such that engagement surfaces 125 are disposed at an acute angle relative to shaft 111. For example, the acute angle formed between each engagement surface 125 and shaft may be 45 degrees to 90 degrees. Stated differently, in the open position, engagement surfaces 125 of distal elements 120 may be oriented 90 degrees to 180 degrees relative to each other. However, it is generally preferable for arms to be positioned 120 degrees relative to each other (and 60 degrees relative to shaft 111) for capturing leaflets. Movement of fixation device 112 from the closed position to the open position may be achieved by advancing stud 131 distally relative to coupling member 160 by distally advancing actuator rod 170. Conversely, fixation device 112 may be moved from the open position to the closed position by retracting actuator rod 170 and retracting stud 131 proximally, according to one example of the disclosure.

As shown in FIG. 10B, proximal elements 140 (or proximal elements 240) may be in a raised or insertion position when fixation device 112 is in the open position to facilitate insertion of leaflets between distal and proximal elements 120, 140 for their capture. A loop 148 may be provided on one or both proximal elements 140 for receipt of a proximal element line that can raise and lower proximal elements 140. Proximal elements 140 are, in one example, biased toward distal elements 120. In this regard, proximal elements 140 may be moved inwardly toward shaft 111 and held against shaft 111 with the aid of proximal element lines 101 which can be in the form of sutures, wires, nitinol wire, rods, cables, polymeric lines, or other suitable structures, as shown in FIG. 10A. Thus, FIGS. 10A and 10B depict fixation device 112 in an insertion configuration in which proximal elements 140 are in a raised position and distal elements 120 are in an open position.

Once fixation device 112 has been positioned in a desired location against the valve leaflets, the leaflets may then be captured between proximal elements 140 and distal elements 120. FIGS. 11A and 11B illustrate fixation device 112 in an example of such a position. Here, proximal elements 140 are lowered toward engagement surfaces 125 so that proximal elements 140 are in a lowered or capture position, and the leaflets are held between distal and proximal elements 120, 140. Proximal elements 140 are, in one example, lowered into the lowered position while distal elements 120 remain in the open position. Thus, fixation device 112, as shown in FIGS. 11A and 11B is in an example of a capture configuration which may be similar to the insertion configuration of FIGS. 10A and 10B, but with the difference being that proximal elements 140 are now lowered toward distal elements 120 by releasing tension on proximal element lines 101 to compress the leaflet tissue therebetween. At any time, the proximal elements 140 may be raised and the distal elements 120 adjusted or inverted to reposition fixation device 112 if regurgitation is not sufficiently reduced according to one example of the disclosure.

FIGS. 12A-12B depict an example of an inverted position of fixation device 112. Fixation device 112 may assume the inverted position to aid in repositioning or removal of fixation device 112. In one example of the inverted position, distal elements 120 may be further rotated from the open position, which may be achieved by advancing stud 131 further relative to the open position, so that the engagement surfaces 125 of distal elements 120 face outwardly, and free ends 121b point distally. Additionally, in some examples, engagement surfaces 125 of each arm 121 may form an obtuse angle relative to shaft 111. For example, the obtuse angle formed between each engagement surface 125 and shaft 111 may be 135 degrees to 180 degrees. Stated differently, in the inverted position, engagement surfaces 125 of distal elements 120 may be oriented 270 degrees to 360 degrees relative to each other.

Also, as shown in FIG. 12B, in one example proximal elements 140 are in their raised position against shaft 111 while distal elements 120 are in the inverted position by exerting tension on the proximal element lines 101. Thus, a relatively large space may be created between proximal and distal elements 140, 18 for repositioning. In addition, the inverted position allows withdrawal of the fixation device 112 through the valve while minimizing trauma to the leaflets. Engagement surfaces 125 provide an atraumatic surface for deflecting tissue as the fixation device is retracted proximally. It should be further noted that tines 145 of proximal elements 140 may, in some examples, be angled slightly in the distal direction (away from the free ends of the proximal elements 140), reducing the risk that tines 145 will catch on or lacerate tissue as fixation device 112 is withdrawn and while proximal elements 140 are in the raised position.

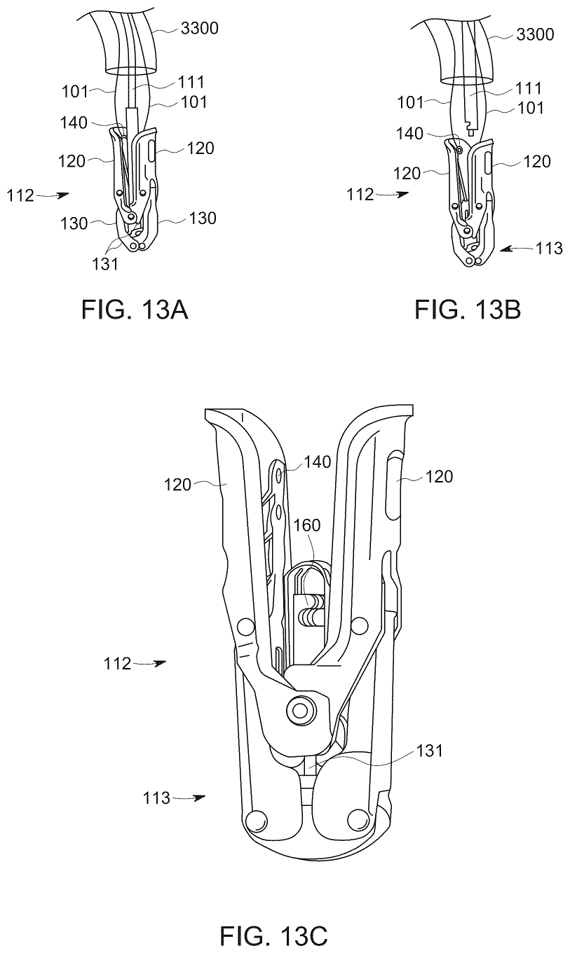

After the leaflets have been captured between distal and proximal elements 120, 140, distal elements 120 may be returned to or toward the closed position where they may be locked in place. An example of such locking is described further below. FIG. 13A illustrates fixation device 112 in the closed position wherein the leaflets (not shown) are captured and coapted. In one example, this is achieved by retraction of the stud 131 proximally relative to coupling member 160 so that the legs 130 of the actuator 113 apply an upwards force to distal elements 120 which in turn rotate distal elements 120 so that engagement surfaces 125 again face one another, similar to that of FIGS. 9A and 9B, and so that distal elements 120 rotate proximal elements 140 in a direction toward shaft 111. However, because the leaflets are captured between distal and proximal elements 120, 140, it may be desirable to keep distal elements 120 at about 20 degrees to 60 degrees relative to each other so as to limit the amount of tension and stress on the native tissue. Thus, while fixation device 112 may be returned to the closed position, such closed position may not be as closed as in the initial delivery position.

As shown in FIG. 13B, fixation device 112 may then be released from shaft 111 of delivery system 600 while in the closed position. As mentioned, fixation device 112 may be releasably coupled to delivery system 600 via a coupling system (e.g., coupling system 115 or 315). When the coupling structures of such coupling system are released, proximal element lines 101 may remain attached to proximal elements 140 following detachment to function as a tether to keep the fixation device 112 connected with the delivery catheter 610 (see FIG. 16) for reconnection and repositioning. However, in other embodiments, proximal elements lines 101 may be released prior to release of fixation device 112 or concurrently with the release of fixation device 112, as described in more detail below.

FIG. 13C illustrates a released fixation device 112 in an example of a closed position. As shown, coupling member 160 remains separated from shaft 111 of delivery system 600, and proximal elements 140 are deployed so that tissue (not shown) may reside between proximal elements 140 and distal elements 120.

As mentioned above, proximal element lines or actuators 101 may be releasably coupled to proximal elements 140. In some examples, proximal element lines 101 may pass through an opening in proximal elements 140, such as openings 146 and 246 in the case of proximal element 240. In other examples, eyelets, which may be formed from one or more lengths of suture, may be coupled to proximal elements 140 and proximal element lines 101 may pass through such eyelets. Thus, proximal element lines 101 may be released from proximal elements 140 prior to, concurrent with, or after release of fixation device 112 from delivery system 600 according to various examples.

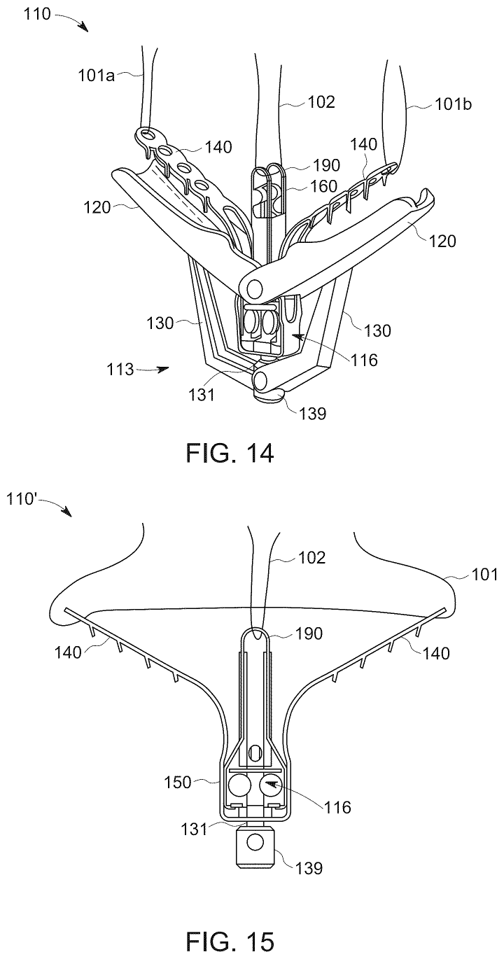

In an exemplary embodiment of interventional tool 110, as shown in FIG. 14, a plurality of proximal element lines 101a, 101b may extend through corresponding lumens 614a, 614b of delivery catheter 610 of delivery system 600 (see FIG. 16) and may be coupled to proximal elements 140 of fixation device 112. Each of proximal element lines 101a and 101b may be elongated flexible threads, wire, cable, sutures, or lines extending through shaft 111, looped through proximal elements 140, and extending back through shaft 111 to a delivery device handle of delivery system 600. When detachment is desired, one end of each proximal element line 101a, 101b may be released from delivery system 600, and the other end pulled to draw the free end distally through shaft 111 and through proximal element 140 thereby releasing it. Also, in this arrangement, proximal element lines 101a and 101b may be independently or concurrently manipulated so as to independently or concurrently raise and lower proximal elements 140, respectively.

In another example, interventional tool 110′ may be configured, as shown in FIG. 15 with respect to certain components thereof, such that proximal elements 140 may alternatively be supported by a single proximal element line 101 which may extend through both of the proximal elements 140. In this arrangement both proximal elements 140 may be raised and lowered concurrently by action of a single proximal element line 101. Whether proximal elements 140 are manipulated individually by separate proximal element lines 101 or jointly by a single proximal element line 101, the proximal element lines 101 may extend directly through openings (e.g., openings 146, 246) of the proximal elements 140 and/or through a layer or portion of a covering 117 on proximal elements 140, or through a suture loop/eyelet above or below a covering 117, for example.

In a further example, interventional tool 110″ may be configured, as shown in FIG. 16, such that each proximal element line 101a, 101b may be releasably engaged with structures that are activated by removal of the actuator rod 170 that passes through coupling member 160 and shaft 111 such that release of proximal element lines 101a, 101b occurs concurrently with the release of fixation device 112 from delivery system 600. Thus, in one example, which is depicted in FIG. 16, each proximal element line 101a, 101b may have a first end portion 103a (e.g., proximal end portion), which may be coupled to an actuator of a delivery system handle, a second end portion 103b (e.g., distal end portion) which may be releasably engages to shaft 111 via actuator rod 170, and an intermediate portion 103c which may be coupled to a proximal element 140. As described above and as illustrated in FIG. 7A, stud 131 may be releasably attached to actuator rod 170 which passes through coupling member 160 and shaft 111 of delivery system 600. In this way, actuator rod 170 is connectable with fixation device 112 and acts to manipulate fixation device 112 so as to move it through its various positions, which are described above. After the leaflets have been coapted, actuator rod 170 may be removed proximally from stud 131 which may thereby also release coupling member 160 from shaft 111, as described with respect to FIGS. 7A-7C and also FIGS. 8A and 8B with respect to coupling system 315. This action of actuator rod 170 may be utilized to release distal end portion 103b of each of proximal element lines 101a, 101b.

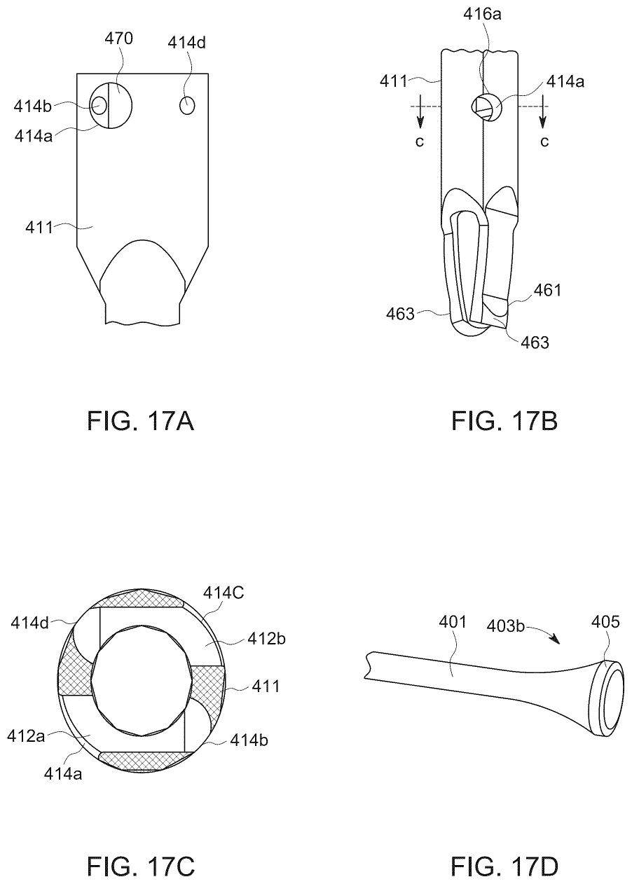

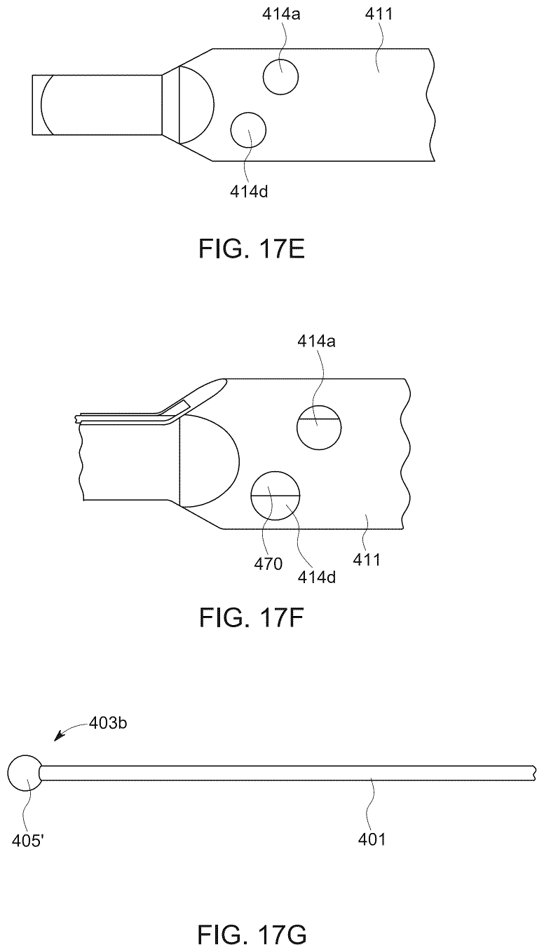

Exemplary features which may be implemented in interventional tool 110″ to facilitate release of proximal element lines 101a, 101b in this manner are shown in FIGS. 17A-17G. As depicted, an actuator rod 470 may be used as an anchor to restrict proximal movement of one or more proximal element lines 401. Proximal element line 401 has a distal end portion 403b which may include a catch element 405, for example a trumpet 405 having a cone shape (see FIG. 17D) or other shapes, such as a ball 405′ having a spherical shape (see FIG. 17G), which can be sized to be received within shaft 411. As shown in the example of FIGS. 17B, shaft 411 may have spring arms 461 like that of the coupling system 315 of FIGS. 8A and 8B for releasing device 112 from shaft 411. However, shaft 411 may also have mating surfaces of FIGS. 7A and 7C. In any event, a portion of shaft 411 proximal of spring arms 461 (or mating elements 463), may have two slots 412a and 412b defined therein. Slot 412a can define holes 414a and 414b and slot 412b can define holes 414c and 414d. Holes 414a and 414c can be sized to receive catch element 405 of a pair of proximal element lines 401, respectively, therethrough and into slots 412a and 412b, respectively. Holes 414b and 414d can be sized to prevent catch element 405 of proximal element lines 401, respectively, from extending beyond slots 412a and 412b, respectively. The configuration of slots 412a and 412b and holes 414a-414d can allow for easier manufacture of the features in shaft 411. Slots 412a and 412b can be drilled to ensure that slots 412a and 412b do not pass the entire way through shaft 411. In this example configuration, catch elements 405 of proximal element lines 401 can be maintained within shaft 411 to manage the slack of proximal element lines 401.

In one example, catch element 405 of proximal element line 401 can be inserted into slot 412a through hole 414a beyond a longitudinal axis of shaft 411 and toward hole 414b, and catch element 405 of proximal element line 401 can be inserted into slot 412b through hole 414c beyond the longitudinal axis of shaft 411 and toward hole 414d prior to the insertion and coupling of the actuator rod 470 (which passes through shaft 411) with stud 131 of fixation device 112. With actuator rod 470 extending through shaft 411, actuator rod 470 may directly engage catch elements 405 of lines a plurality of proximal element lines 401 thereby preventing their movement back out along the path through which they were inserted. For example, trumpets 405 can be inhibited from being advanced through holes 414b and 414d, respectively, and can be prevented from being pulled past actuator rod 470 and through holes 414a and 414c, respectively. Accordingly, the second end portions 403b of proximal element lines 401 can be held in place relative to shaft 414. Once the actuator rod 470 is decoupled from stud 131 and subsequently retracted, movement of catch elements 405 at the distal end portions of proximal element lines 401 is no longer restricted and proximal element lines 401 are free to move. Upon proximal retraction, proximal element lines 401 can thread through holes 414a and 414c, respectively, and decouple from the proximal elements 140.

In accordance with one example of the disclosed subject matter, slots 412a and 412b can be drilled at an angle towards the distal end of shaft 411 (see FIGS. 17E and 17F), e.g., with hole 414b formed distal to hole 414a on one side, and hole 414d formed distal to hole 141c on the other side. This example configuration of slots 412a and 412b can provide easier deployment of a plurality of proximal element lines 401 and can reduce friction.

Prior to securing second end portion 403b of each proximal element line 401 with the shaft 411, each proximal element line 401 can be coupled with a respective proximal element 140, such as in the manner described above with respect to FIG. 16. Thus, when proximal element lines 401 are actuated proximally, proximal element lines 401 can move proximal elements 140 relative to distal elements 120, thereby moving proximal elements 140 between their respective raised and lowered positions.

As mentioned above, fixation device 112 optionally includes a lock (e.g., lock 116) for locking device 112 in a particular position, such as in any one of the aforementioned open, closed, and inverted positions or any position therebetween. It may be appreciated that according to various examples, lock 116 may be configured for both locking and unlocking which correspondingly allows device 112 to be both locked and unlocked. As described in more detail below with respect to various lock examples, such locks may have components disposed between coupling member 160 and base 139 which may be configured to selectively arrest proximal-distal movement of stud 131/base 139 which consequently arrests movement of distal elements 120. Such locks may help provide end user control of the final arm angle of fixation device 112 for tailored and optimal results for each patient. Additionally, such locks may bring the leaflets and annulus together which may result in beneficial dimensional changes of the target valve which can prevent adverse remodeling of the heart, particularly for patients with heart failure.

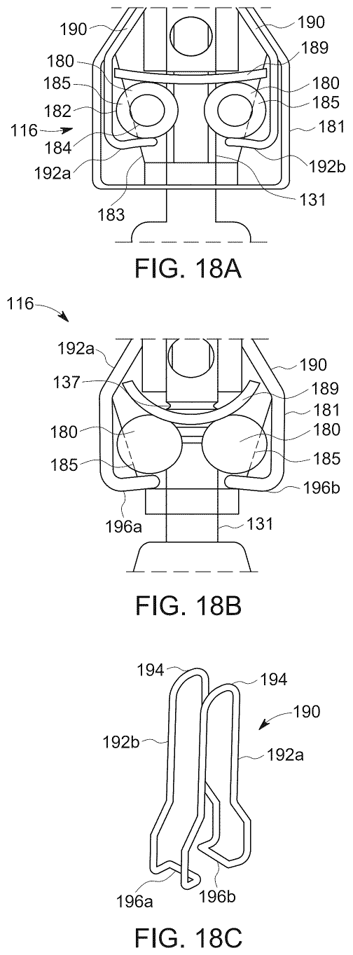

FIGS. 14, 15, and 16A-16C illustrate an embodiment of the lock 116. Lock 116 generally includes a housing 181, one or more wedging elements 180, a release harness 190, and a biasing member 189. Housing 181 may be positioned distal to coupling member 160 and may be free-floating, coupled to, or integral with coupling member 160, such as at a distal end thereof. Housing 181 may form a window 183 which may be defined at opposite sides with sloping or tapered surfaces 185 which slope inwardly toward stud 131 in a proximal to distal direction. Wedging elements 180 may be in the form of rolling elements, such as a pair of barbells, disposed on opposite sides of stud 131 and between sloping surfaces 185, as shown in FIGS. 18A and 18B. Each barbell 180 may have a pair of generally cylindrical caps 182 and a shaft 184 therebetween, as illustrated in the barbell cross-section of FIG. 16A. Barbells 180 and stud 131 are preferably comprised of cobalt chromium or stainless steel, however any suitable material may be used. Biasing member 189 may be a spring, such as a leaf spring, for example, and may be positioned at a proximal end of housing 181 between sloping surfaces 185 and proximal to barbells 180 such that spring 189 bears on barbells 180 and biases them in a distal direction. Thus, when barbells 180 are pushed distally by spring 189, they are correspondingly pushed inwardly and wedged against stud 131 by sloping surfaces 185, as illustrated by FIG. 18A, which depicts barbells 180 in a proximal and unlocked position, and FIG. 18B, which depicts barbells 180 in a distal and locked position.

As shown in FIGS. 14, 15, and 18C, release harness 190 may be in the form of a ridged wire or rod that may extend proximally from stud 131 toward a proximal end of fixation device 112 and at opposite sides thereof. In this regard, release harness 190 may form a first portion or front portion 192a and a second portion or rear portion 192b. Each of first and second portions 192a, 192b may include a crest or closed proximal end 194 through which a lock line 102 may be threaded and engaged, as described below. Release harness 190 may also form hooked distal ends 196a, 196b which may extend between first and second portions 192a, 192b and between sloping surfaces 185 and stud 131, as shown in FIGS. 18A and 18B. Thus, hooked ends 196a and 196b may be moveable proximally-distally within window 183 formed between sloped surfaces 185 and stud 131. Additionally, hooked ends 196a and 196b may be positioned distal of barbells 180 such that pulling up on harness 190 moves hooked ends 196a, 196b proximally so as to push the respective barbells 180 against the bias of spring 189 and move them to their unlocked position.

Movement of harness 190 may be performed by one or more lock line 102 which may be coupled to harness 190 by such as by threading lock line 102 through and engaging one or more of proximal ends 194 of first and second portions 192a, 192b thereof, as shown in FIGS. 14 and 15. Such lock line 102 may have a first end 102a fixedly secured to a delivery system handle 1012 of delivery system 600 and a second end 102b releasably secured to a delivery system handle 1012, as described in more detail below. In this regard, tension can be selectively applied to lock line 102 to unlock and lock the lock 116. Also, lock line 102 can be released from release harness 190 prior to, concurrently with, or after release of fixation device 112 from delivery system 600 which may be achieved by releasing the second end 102b from delivery system handle and pulling lock line 102 and its second end through shaft 111. Lock line 102 may be comprised of any suitable material, typically wire, nitinol wire, cable, suture, or thread, to name a few. In addition, lock line 102 may include a coating, such as parylene. Parylene is a vapor deposited pinhole free protective film which is conformal and biocompatible. It is inert and protects against moisture, chemicals, and electrical charge.

When an upwards force is applied to harness 190 by the lock line 102, hooked ends 196a, 196b may raise barbells 180 against spring 189, as shown in FIG. 18A. This may draw barbells 180 up along sloping surface 185 which unwedges barbells 180 from against stud 131. In this position, stud 131 is free to move. Thus, when lock line 102 is tensioned to raise or lift harness 190, lock 116 is in an unlocked position wherein stud 131 is free to move actuator 113 and therefore distal elements 120 to any desired position. Releasing tension in lock line 102 may, on the other hand, transition the lock 116 to a locked position, as shown in FIG. 18B. Thus, by releasing the upwards force on barbells 180 by hooked ends 192a, 192b, spring 189 forces barbells 180 downwards and wedges barbells 180 between a sloping surface 185 and stud 131. This restricts motion of stud 131, which in turn locks actuator 113 and therefore distal elements 120 in place. In addition, stud 131 may include one or more grooves or indentations 137 which may receive shaft 184 of each barbell 180. This may provide more rapid and positive locking by causing barbells 180 to settle in a definite position, increase the stability of lock 116 by further preventing movement of barbells 180, as well as tangible indication to the user that each barbell 180 has reached a locking position. In addition, grooves 137 may be used to indicate the relative position of distal elements 120, particularly the distance between distal elements 120. For example, each groove 137 may be positioned to correspond with a 0.5- or 1.0-mm decrease in distance between distal elements 120. As stud 131 is moved, barbells 180 may contact grooves 137, and by counting the number of grooves 137 that are felt as stud 131 is moved, the user can determine the distance between distal elements 120 and can provide the desired degree of coaptation based upon leaflet thickness, geometry, spacing, blood flow dynamics and other factors. Thus, grooves 137 may provide tactile feedback to the user.

Lock 116 allows fixation device 112 to remain in an unlocked position when attached to delivery system 600 during grasping and repositioning and then maintain a locked position when left behind as an implant. It may be appreciated, however, that lock 116 may be repeatedly locked and unlocked throughout the placement of the fixation device 112 if desired. Once the final placement is determined, lock line 102 may be removed and fixation device 112 may be left behind.

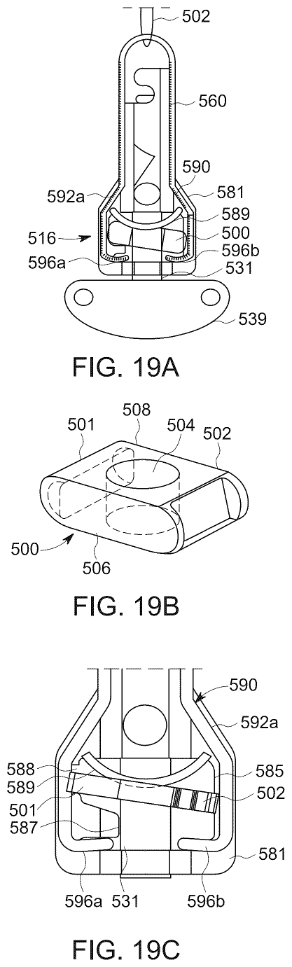

FIGS. 19A-19C depict a lock 516 according to another embodiment of the present disclosure that may be incorporated into a fixation device 112 of the disclosure. In this embodiment, lock 516 also includes a housing 581, a spring 589, a release harness 590, and a wedging element 500. However, instead of sloping surfaces 185 as present in the example of locking element 116, housing 185 may include generally parallel sidewalls 585 and may include a finger or protrusion 587 extending from one of sidewalls 585 toward stud 131, as best shown in FIG. 19C. Such finger 587 may slope in a distal direction and may define a proximal notch 588. Also, as shown in FIG. 19C, first hooked end 596a of release harness 590 may be positioned distal of finger 587.

Furthermore, wedging element may comprise a binding lever or binding plate 500. As shown in FIG. 19B, binding plate 500 may have an oblong shape that may extend lengthwise between a first end 501 and a second end 502 thereof. An aperture 504 may be formed between first and second ends 501, 502 and may extend from a top planar surface 508 through a bottom planar surface 506 of binding plate 500. Binding plate 500 may be positioned between sidewalls 406 so that stud 131 passes through aperture 504 and so that first end 501 of binding plate 500 is positioned within notch 588 proximal of finger 587, as best shown in FIG. 19C. Thus, finger 587 may be positioned between first end 501 of binding plate 500 and first hooked end 596a of released harness 590. Also, spring 589 may be positioned proximal to binding plate 500 and provide downward or distal bias thereto. Binding plate 500 and stud 131 may be comprised of any suitable material. In some embodiments, binding plate 500 may have a higher hardness than stud 131. In other embodiments, binding plate 500 may be comprised of a flexible or semi-flexible material. Such flexibility may allow slight movement of stud 131 in the proximal and distal directions, therefore allowing slight movement of distal elements 120 when lock 516 is in the locked position. This may allow fixation device 112 to adjust in response to dynamic cardiac forces.

FIGS. 19A and 19C illustrate binding plate 500 in a locked position or configuration. In this regard, spring 589 pushes binding plate 500 in a distal direction. However, because first end 501 of binding plate 501 is positioned within notch 588, axial movement of first end 501 toward a distal end of housing 581 is prohibited while axial movement of second end 502 of binding plate 500 is permitted. Thus, finger 587 obstructs first end 501 from axial movement and creates a lever type movement of binding plate 500. Moreover, finger 587 obstructs first hooked end 596a of release harness 590 from axial movement resulting in a side-to-side pivoting of release harness 590 upon tension of lock line 102. This pivoting movement correspondingly results in second hooked end 596b of release harness moving proximally and controlling movement of second end 502 of binding plate 500. As such, when an upwards force is applied to harness 590 by lock line 102, second hooked end 596b of release harness 590 raises second end 502 of plate 500 against spring 589 so that planar surfaces 506, 508 of binding plate 500 become oriented substantially perpendicular to stud 131. This aligns aperture 504 with stud 131 allowing free movement of stud 131 in the proximal-distal direction. Thus, in this state, lock 516 is unlocked wherein stud 131 is free to move actuator 113 and therefore distal elements 120 to any desired position.

Release of harness 590 by lock line 102 transitions lock 516 back to the locked position. By releasing the upwards force on second end 502 of binding plate 500, spring 589 forces second end 502 of biding plate 500 downwards, which misaligns aperture 504 relative to stud 531, and correspondingly wedges binding plate 500 against stud 131, as best shown in FIG. 19C. This arrests movement of stud 131, which in turn locks actuator 113 and therefore distal elements 120 in place. It may be appreciated that binding plate 500 may have any suitable form to function as described above. For example, plate 500 may have a variety of shapes with or without planar surfaces 506, 508 and/or the aperture 504 may be of a variety of shapes and positioned in a variety of locations, to name a few. For example, binding plate 500 may not have a through-hole, like that of aperture 504, but may rather have a notch such that binding plate 500 does not encircle stud 131 but rather partially surrounds it. Further, it may be appreciated that any number of binding plates 500 may be present. Each binding plate 500, in this regard, may provide an additional binding location which may enhance lock performance.

While the above-described nonlimiting examples of fixation device 112 may utilize a push-to-open, pull-to-close mechanism for opening and closing distal elements 120, it should be understood that a pull-to-open, push-to-close mechanism may alternatively be provided. For example, distal elements 120 may be coupled at their proximal ends to stud 131 rather than to coupling member 160, and legs 130 may be coupled at their proximal ends to coupling member 160 rather than to stud 131. In this example, when stud 131 is pushed distally relative to coupling member 160, distal elements 120 may close, while pulling on stud 131 proximally toward coupling member 160 may open distal elements 120. Regardless, the aforementioned lock examples may be configured to arrest stud to lock distal elements 120 in the desired position, as described.

It is to be understood that the fixation devices and components thereof described above are provided as examples are not to be considered as limiting to fixation devices suitable for use with other aspects of the disclosure.

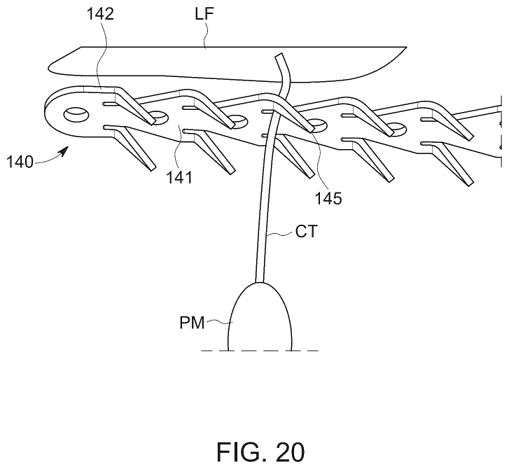

During a TEER procedure, a fixation device, such as fixation device 112, may unintentionally snag or engage anatomic tissue structures, such as chordae tendineae CT, papillary muscles PM, or leaflets LF, for example. An exemplary situation in which this may occur is depicted in FIG. 20 with respect to proximal element 140 of fixation device 112. As described above, proximal element 140 may have frictional elements 145 formed from side edges 142 of elongate arm 141. When fixation device 112 is being positioned within a valve, chordae CT or other anatomic structures may become caught or snagged on a frictional element 145 or in a space between frictional elements 145. This undesirable engagement of tissue can hinder the TEER procedure and present difficulty to the operator as they must first disengage the tissue before properly placing fixation device 112 in an optimal location on the valve.

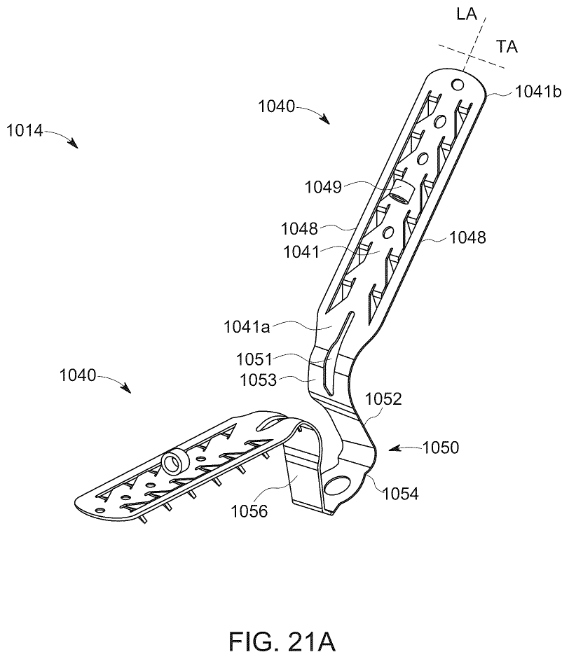

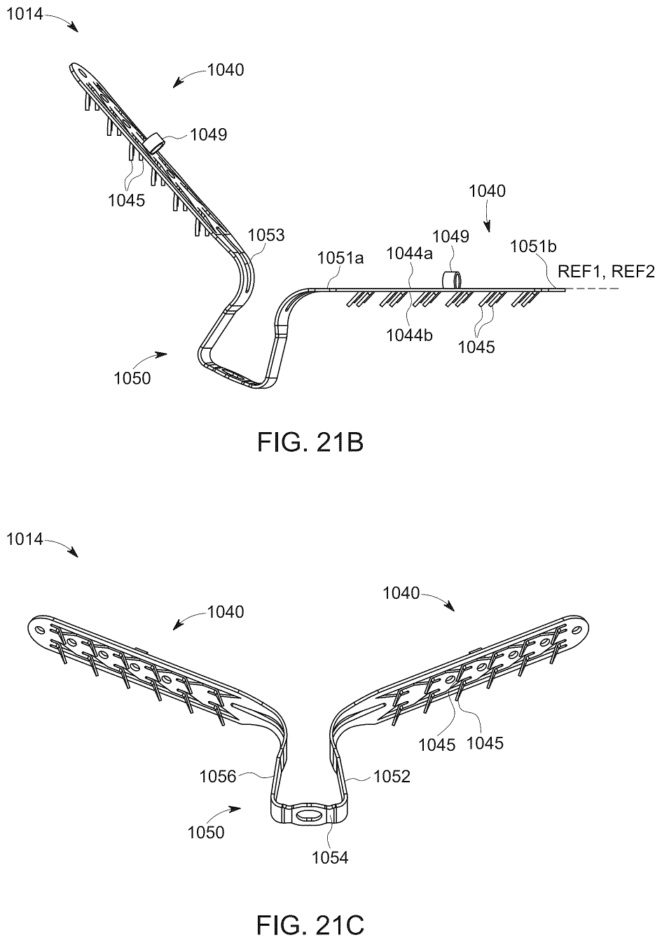

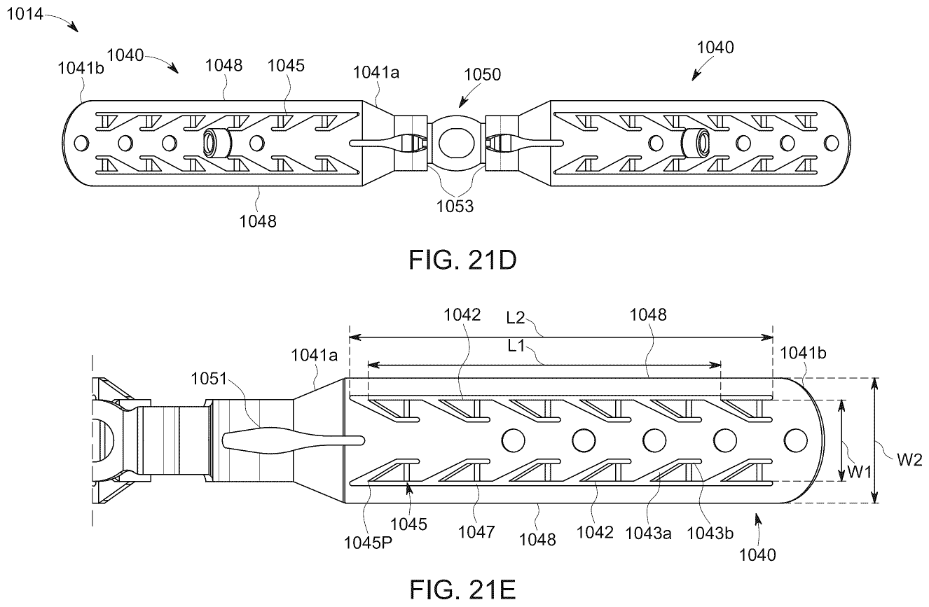

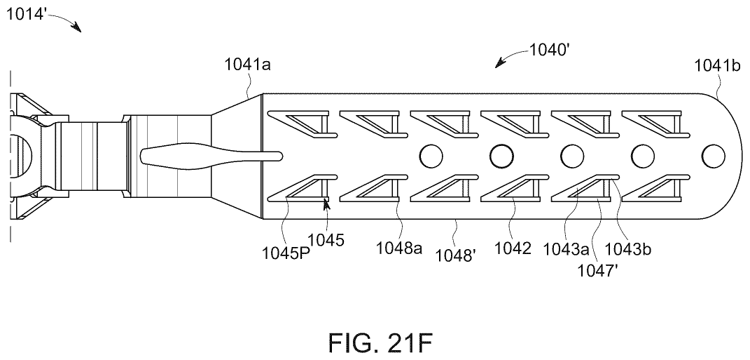

Referring now in addition to FIGS. 21A-21D, which depict a gripping device 1014 according to another embodiment of the present disclosure, which may be incorporated into any of the fixation devices of the disclosure. Gripping device 1014 is configured to mitigate or prevent unintentional interaction with and snagging of tissue, such as the chordae CT, papillary muscles PM, and leaflets LF, for example.

Gripping device 1014 is similar to gripping device 114 except for differences explicitly described and/or shown and may include any features or characteristics of any other gripping device of the disclosure. Thus, like elements are accorded like reference numerals to that of gripping device 114 but within the 1000-series of numbers, meaning base section 1050 is alike to base section 150, arm bend features 1053 are alike to arm bend features 153, and so on, except for differences explicitly described and/or shown. In addition to base section 1050 and arm bend features 1053, gripping device 1014 generally includes a pair of proximal elements 1040.

As shown, each proximal element 1040 may include an elongate arm 1041 that has a first end portion or fixed end 1041a and a second end portion or free end 1041b. First end portion 1041a may be connected to and extend from a corresponding arm bend feature 1053 which may be connected to and extend from a base section 1050. Arm bend features 1053 and base section 1050 are similar to arm bend features 153 and base section 150 of gripping device 114, which is described above. Elongate arm may also include a loop 1049 which may be configured to receive a proximal element line, such as line 101, for actuating proximal element 1040 from a raised and lowered position. As shown, loop 1049 may be positioned at or near a midpoint of elongate arm 1041. However, in other embodiments, loop 1049 may be positioned at other locations along its length, such as at or adjacent to free end 1041b.

Elongate arm 1041 may be similar to elongate arm 141 in that it may include opposing side edge 1042 each forming a plurality of frictional elements 1045 (only one of which is referenced for case of illustration), such as by cutting and bending the base material forming proximal elements 1040, for example. In this regard, frictional elements 1045 may be integral with elongate arm 1041 so as to form a monolithic structure. As shown in FIG. 21E, the formation of each frictional element 1045 from a side edge 1042 may form one or more corresponding notches or openings. For example, a first notch or outer notch 1043a (also referred to herein as first opening or inner opening) may extend inwardly from a side edge in a direction toward a longitudinal axis LA of elongate arm 1041, and a second notch or inner notch 1043b (also referred to herein as second opening or inner opening) may intersect first notch 1043a and extend in a direction along longitudinal axis LA. First notch 1043a may be angled relative to longitudinal axis LA or may be perpendicular to longitudinal axis LA, and second notch 1043b may be parallel (+/−5 degrees) relative to longitudinal axis LA or may be angled relative to longitudinal axis LA, for example. Such notches 1043a, 1043b may together form the shape of a frictional element 1045 which may be bent in a distal direction such that it extends distally from a distal surface 1044b of proximal element 1040. Additionally, each frictional element 1045 may terminate at a prong 1045P or a plurality of prongs which may be configured for atraumatic frictional engagement.

Each proximal element 1040 may include a plurality of frictional elements 1045 which may be arranged in rows of one or more frictional elements 1045. For example, proximal element 1040 may include one to six rows of frictional elements 1045, and each row may include one to four frictional elements 1045 aligned along a transverse axis TA. In the embodiment depicted, each proximal element 1040 includes six rows of frictional elements 1045 with each row having two frictional elements 1045 formed from opposing side edges 1042 and each defining first and second notches 1043a, 1043b. The rows of frictional elements 1045 may be arranged at consistent or varying intervals along a first length L1. In other words, the rows of frictional elements 1045 may be distributed along first length L1 as measured from prongs 1045P of frictional elements 1045 at the end rows closest to first and second end portions 1041a, 1041b. For example, as shown in FIG. 21E, first length L1 extends from a first row closest to first end portion 1041a to a sixth row closest to second end portion 1041b.

As mentioned above, tissue may become snagged by one of frictional elements 1045 or within one of the notches 1043a, 1043b between them during a TEER procedure. To help prevent or mitigate tissue from becoming snagged, proximal element 1040 may include a pair of guard rails or axial struts 1048. Guard rails 1048 may be connected to first end portion 1041a and second end portion 1041b and extend therebetween and along a corresponding side edge 1042 so as to effectively enclose notches 1043a and 1043b and block tissue from entering notch 1043a and/or 1043b and/or becoming snagged on a frictional element 1045. As shown in FIG. 21E, guard rails 1048 may each extend along a second length L2 which may be greater than first length L1 such that guard rails 1048 extend beyond all rows of frictional elements 1045. However, in some embodiments, each guard rail 1048 may instead be connected to a side edge 1042 of elongate arm 1041 and extend along less than all rows of frictional elements 1045, for example. Each guard rail 1048 may extend parallel (+/−5%) relative to longitudinal axis LA and may be offset from side edges 1042 so as to form an elongate opening 1047 between side edge 1042 and guard rail 1048. Such elongate opening 1047 may be in communication with first notch 1043a of each frictional element 1045. Also, as shown in FIG. 21B, elongate arm 1041 may define a reference plane REF1, and each guard rail 1048 may define a second reference plane REF2. Guard rails 1048 may be flush with elongate arm 1041 (i.e., at the same elevation as elongate arm 1041) such first reference plane REF1 is coplanar with second reference plane REF2 of each guard rail 1048.

In addition to preventing tissue from becoming snagged by proximal element 1040, guard rails 1048 may also increase the stiffness of proximal element 1040 which may enhance tissue retention. Also, as shown in FIG. 21E, elongate arm 1041 may have a first width W1 and guard rails 1048 collectively may define a second width W2 greater than first width L1. Thus, guard rails 1048 increase the width of proximal element 1040 which may further enhance tissue retention. In some embodiments, frictional elements 1045 may be formed from guard rails 1048, such as by cutting guard rails 1048 from an inner edge thereof so as to retain the protective benefits of guard rails 1048, while enhancing desirable tissue engagement. In such embodiments, guard rails 1048 may have a wider individual width than that shown in FIG. 21E to provide further standoff distance from its outer edge and the frictional elements 1045 extending therefrom.

Guard rails 1048 may be separately connected to first and second end portions 1041a, 1041b, such as by welding, for example. Guard rails 1048 may also be formed from the same base material as elongate arm 1041 and frictional elements 1045, such as by cutting (e.g., laser cutting) the base material to form guard rails 1048. In this regard, proximal elements 1040 may be made from a memory metal material, such as nitinol, for example. A blank of such memory metal material may be cut to form frictional elements 1045 and guard rails 1048. In other embodiments, guard rails 1048 may be made from a polymeric material, such as poly-L-lactide (“PLLA”), poly lactic-co-glycolic acid (“PLGA”), polyetheretherketone (“PEEK”), acrylonitrile butadiene styrene (“ABS”) or polyurethane, for example. Guard rails 1048 may also be made from a metal material, such as titanium, cobalt-chromium, or stainless steel, for example. Further, guard rails 1048 may be made from a foam-like material which may incorporate a hydrogel, for example.

Gripping device 1014 may be utilized in conjunction with distal elements 120 of fixation device 112, as an example. Additionally, while proximal element 1040 is depicted as being connected to and extending from arm bend feature 1053 and base section 1050, which are similar to arm bend feature 153 and base section 150 of FIGS. 5A and 5C, it should be understood that proximal element 1040 can be connected to and extend from arm bend feature 260 and second arm 250 of FIGS. 6A and 6B or bend feature 260 and second arm 250′ of FIG. 6C, for example. Thus, proximal element 1040 may be adapted to couple to a center portion of a fixation device, like in fixation device 112, or adapted to couple directly to a distal element of a fixation device as has been described with respect to gripping devices 214 and 214′ of FIGS. 5A-5B and 6A-6C.

While each guard rail 1048 is depicted as forming an elongate opening 1047 that intersects each first notch 1043a formed by a corresponding frictional element 1045 at one side 1042 of elongate arm 1041, in other embodiments guard rail 1048 may be connected to a respective side edge 1042 at a plurality of locations. For example, FIG. 21F depicts a proximal element 1040′ of a gripping device 1014′ in which each guard rail 1047′ of proximal element 1040′ may be connected to a respective side edge 1042 at a plurality of locations via a plurality of connection members 1048a extending in a transverse direction from guard rail 1048 to side edge 1042. Such connection members 1048a may interrupt elongate opening so as to form a plurality of elongate openings 1047′ each intersecting a corresponding first notch 1043a. Additionally, each connection member 1048′ may be connected to a side edge 1042 at an end of a respective frictional element 1045 opposite prong 1045P, as shown in FIG. 21F. Such connection members 1048a may enhance the rigidity of proximal element 1040′ and frictional elements 1045.

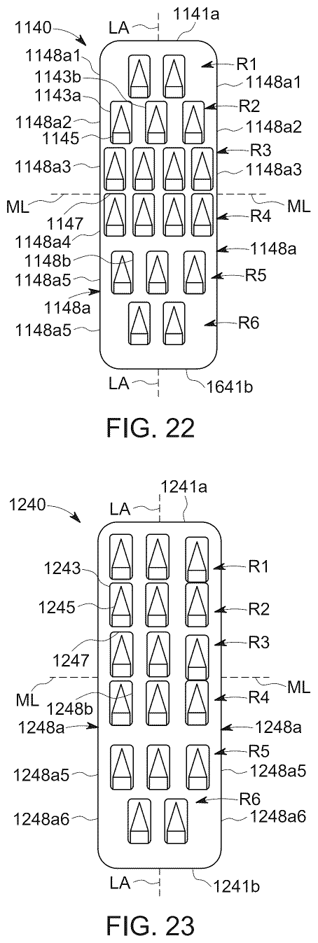

FIG. 22 depicts another example of a proximal element 1140, which may be incorporated into any of the fixation devices of the disclosure and include any of the characteristics and features of other proximal elements disclosed except as explicitly stated. Proximal element 1140 includes rows of frictional elements 1145 closer to fixed end 1141a and free end 1141b have less frictional elements 1145 than rows of frictional elements 1145 closer to a midline ML of proximal element 1140. For example, proximal element 1140 may have six rows of proximal elements 1145 such that a first row R1 is closest to fixed end 1141a, a sixth row R6 is closest to free end 1141b, and third and fourth rows R3, R4 are adjacent to and closest to midline ML. First row R1 and sixth row R6 may each include two frictional elements 1145, and third and fourth rows R3, R4 may each include four frictional elements 1145, for example. Additionally, a second row R2 may be located between first and third rows R1, R3, and a fourth row R4 may be located between fourth and sixth rows R4, R6. Second row R2 and fourth row R4 may each include three frictional elements 1145, for example. Thus, proximal element 1140 exemplifies a frictional element arrangement in which each successive row extending from midline ML may include one less frictional element 1145 than an adjacent row. Other embodiments reflecting a similar configuration may instead include one frictional element 1145 at rows R1 and R6, two frictional elements 1145 at rows R2 and R4, and three frictional elements at rows R3 and R4, for example. In further embodiments in which less than six rows of frictional elements 1145 are provided, such as four rows, for example, the end rows may have two or three frictional elements 1145, while the middle rows closest to midline ML may have three to four frictional elements 1145, for example. Thus, as shown in FIG. 22, the increased frictional element density at midline ML enhances fixation for leaflets inserted to a 50% leaflet insertion depth which may help mitigate incidence of single leaflet device attachment (“SLDA”).

Additionally, proximal element 1140 may have a gridded frame constructed of a plurality of intersecting axial struts or rails 1148a, 1148b and transverse struts or rails 1147. Axial rails 1148a, 1148b may include outer rails 1148a and inner rails 1148b. Inner and outer rails 1148a, 1148b may intersect transverse rails 1147 which together may define a plurality of openings or notches 1143a, 1143b with each opening 1143a, 1143b containing a frictional element 1145 which may be formed from and extend from a corresponding transverse rail 1147. Thus, like proximal elements 140 and 1040, frictional elements 1145 may be formed from the same base material as axial rails 1148a, 1148b and transverse rails 1147, such as by cutting and bending the base material, for example. Openings 1143a, 1143b may include outer openings 1143a and inner openings 1143b. Inner openings 1143b may be defined by opposed inner rails 1148b, and outer openings 1143a may be defined by an outer rail 1148a and an opposed inner rail 1148b. Outer openings 1143a may be disposed at opposed first and second sides of proximal element 1140 while inner openings 1143b may be disposed therebetween. Outer rails 1148a may extend along the first and second sides of proximal element 1040 and may therefore form guard rails which may intersect and close off outer openings 1143a to prevent tissue from being caught therein.

As shown in FIG. 22, outer rails 1148a may each have a variable width along their respective lengths. For example, each outer rail 1148a may have a plurality of rail portions 1148a1-1148a6 with each rail portion 1148a1-1148a6 being adjacent to a respective outer opening 1143a and frictional element 1145. Each rail portion 1148a1-1148a6 may have a width which may be dependent on the number of frictional elements 1145 in an adjacent row R1-R6 of frictional elements 1145. For example, as shown in FIG. 22, first row R1 of frictional elements 1145 is bounded at lateral sides by a pair of first guard rail portions 1148a1, second row R2 of frictional elements 1145 is bounded at lateral sides by a pair of second guard rail portions 1148a2, and third row R3 of frictional elements 1145 is bounded at lateral sides by a pair of third guard rail portions 1148a3. First row R1 of frictional elements 1145 has less frictional elements 1145 than second row R2, and correspondingly first guard rail portions 1148a1 each have a greater individual width than that of second guard rail portions 1148a2. Similarly, second row R2 of frictional elements 1145 has less frictional elements 1145 than third row R3, and correspondingly the pair of second guard rail portions 1148a2 each have a greater individual width than that of third guard rail portions 1148a3. These guard rail portions 1148a1-1148a6 may be interconnected so as to form a continuous outer guard rail 1148a with a variable width. Such configuration may allow proximal element 1140 to have a uniform width along at least a portion of its length while having rows of frictional elements 1145 with differing numbers of frictional elements 1145.

Referring now in addition to FIG. 23, which depicts an additional example of a proximal element 1240, which may be incorporated into any of the fixation devices of the disclosure and include any of the characteristics and features of other proximal elements disclosed except as explicitly stated. Proximal element 1240 includes rows of frictional elements 1245 (only one of which is referenced for ease of illustration) closer to fixed end 1241a and a midline ML of proximal element 1240 may have more proximal elements 1245 than that closest to free end 1241b. For example, proximal element 1240 may have six rows of proximal elements 1245 with a first row R1 being closest to fixed end 1241a and a sixth row R6 being closest to free end 1241b. In the embodiment depicted first row R1, second row R2, third row R3, fourth row R4, and fifth row R5 may each include three frictional elements 1245, while sixth row R6 may include two frictional elements 1745. Third and fourth rows R3, R4 may be closest to midline ML. In other embodiments reflecting a similar configuration, rows R1-R5 may each include four frictional elements 1245, and sixth row R6 may have two to three frictional elements 1245. In further embodiments, fifth and sixth rows R5, R6 may have one to two frictional elements 1245 while rows R1-R4 may each include three to four frictional elements 1245. Thus, as shown in FIG. 23, the increased density of frictional elements 1245 at midline ML helps enhance fixation of leaflets at a 50% insertion depth. Additionally, leaflets tend to have thicker leaflet tissue at their free edge and thinner tissue closer to the valve annulus. The increased density of frictional elements 1245 adjacent fixed end 1241a may help capture this thicker tissue at a full depth of leaflet insertion, while the lower density rows adjacent to free end 1241b may help protect the thinner leaflet tissue.

Additionally, proximal element 1240 is similar to proximal element 1140 in that it may have a gridded frame constructed of a plurality of intersecting inner and outer axial rails 1248a, 1248b and transverse rails 1247 defining a plurality of inner and outer openings 1243a, 1243b. Outer rails 1148a may be guard rails which, similar to proximal element 1140, may have variable widths depending on the number of proximal elements 1245 within any one row of frictional elements 1245. Thus, in this example, sixth row R6 of frictional elements 1245 has less frictional elements 1245 than fifth row R5, and correspondingly a sixth guard rail portion 1248a6, which is aligned with sixth row R6, has a greater width than a fifth guard rail portion 1248b, which is aligned with fifth row R5. However, the individual widths of guard rail portions corresponding to rows R1-R5 each have the same width as each have the same number of frictional elements in each of these rows of frictional elements.

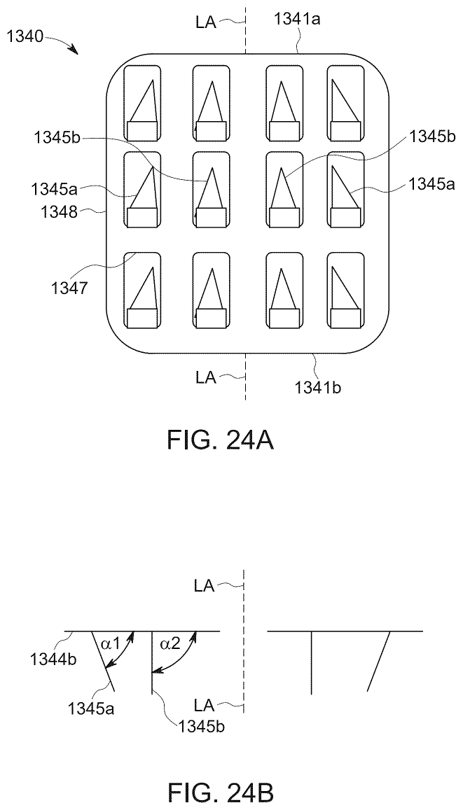

Referring now in addition to FIGS. 24A and 24B, which depict another example of a proximal element 1340, which may be incorporated into any of the fixation devices of the disclosure and include any of the characteristics and features of other proximal elements disclosed except as explicitly stated. Proximal element 1340 includes frictional elements 1345 (only one of which is referenced for ease of illustration) in each row of frictional elements 1345 tilt inwardly toward a longitudinal axis LA of proximal element 1340. For example, a row of frictional elements 1345 may include a pair of outer frictional elements 1345a tilting inwardly at a first tilt angle α1 and a pair of inner frictional elements 1345b tilting inwardly at a second tilt angle α2. Tilt angles α1, α2 are defined between an axis of its respective frictional element 1345 and a distal surface 1344b of proximal element 1340, as shown in FIG. 24B. First and second tilt angles α1, α2 may be equal, for example. In another example, first and second tilt angles α1, α2 may differ such that second tilt angle α2 is greater than first tilt angle α1. For example, as shown, second tilt angle may be 90 degrees while first tilt angle may be less than 90 degrees. Thus, in this example, outer frictional elements are tilted inwardly while inner frictional elements are not. The inward tilt of frictional elements 1345a, 1345b, particularly of outer frictional elements, may help prevent inadvertently snagging tissue that contacts a side of proximal element.

In addition to tilting frictional elements inwardly, outer frictional elements may have different heights than inner frictional elements. For example, inner frictional elements may have a greater height (i.e., may be longer) than outer frictional elements which can help prevent inadvertent tissue engagement particularly of the outer frictional elements which are more likely to snag tissue particularly tissue engaging a lateral side of proximal element.

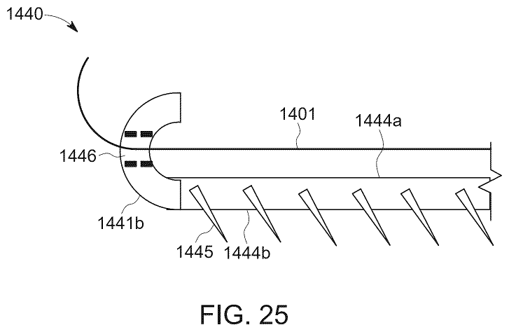

Referring now in addition to FIG. 25, which depicts a proximal element 1440 according to another embodiment of the present disclosure, which may be incorporated into any of the fixation devices of the disclosure and include any of the characteristics and features of other proximal elements disclosed except as explicitly stated. For example, proximal element 1440 may be similar to proximal element 140 or proximal element 1040 with the exception that each frictional element 1445 along the length of proximal element 1440 may be angled relative to a distal surface 1444b at the same angle and in a direction away from free end 1441b. Only one frictional element 1445 is shown for case of illustration. It is to be understood that each frictional element 1445 may be identically configured or may be differently configured in accordance with the disclosure. Additionally, free end 1441b of proximal element 1440 may be rolled upwardly or proximally such that an opening 1446 extending therethrough may receive of a proximal element line 1401, which may be like proximal element line 101, for example. Rolling free end 1441b of proximal element 1440 in this manner may help prevent a leaflet from becoming caught behind proximal element 1440. As indicated, it should be understood that this rolled-end configuration may be utilized in all proximal elements described herein.

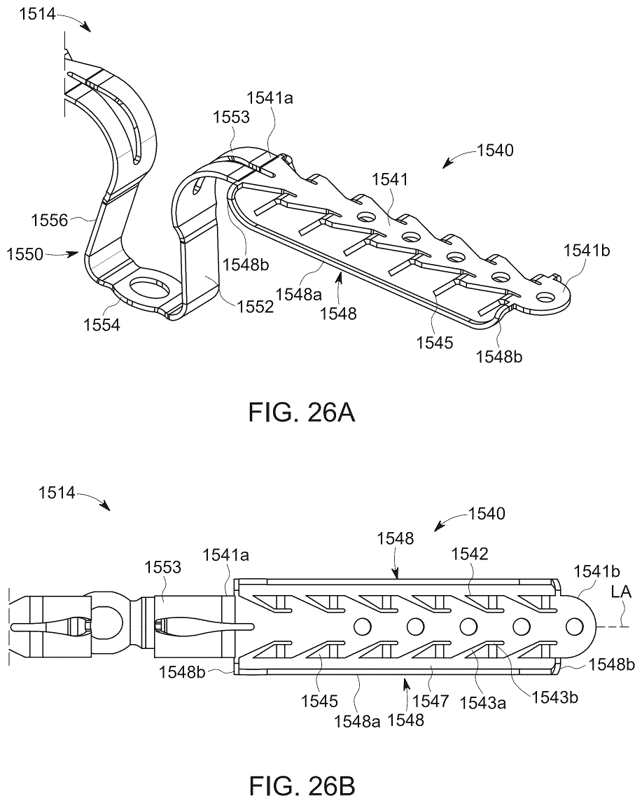

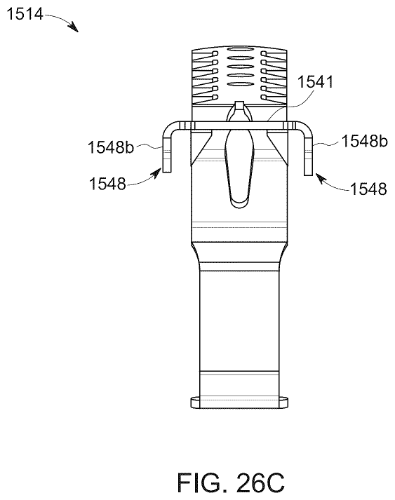

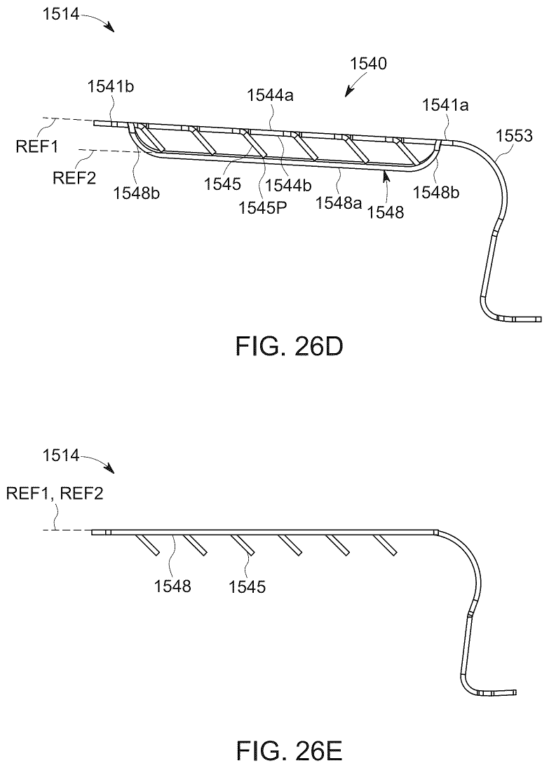

Referring now in addition to FIGS. 26A-26E, which depict a gripping device 1514 according to another embodiment of the present disclosure, which may be incorporated into any of the fixation devices of the disclosure. Gripping device 1514 is also configured to mitigate or prevent unintentional interaction with and snagging of tissue, such as the chordae CT, papillary muscles PM, and leaflets LF, for example.

Gripping device 1514 is similar to gripping device 114 except for differences explicitly described and/or shown and may include any features or characteristics of any other gripping device of the disclosure. Thus, like elements are accorded like reference numerals to that of gripping device 114 but within the 1500-series of numbers, meaning base section 1550 is alike to base section 150, arm bend features 1553 are alike to arm bend features 153, and so on, except for differences explicitly described and/or shown. In addition to base section 1550 and arm bend features 1553, gripping device 1514 generally includes a pair of proximal elements 1540.

As shown, each proximal element 1540 may include an elongate arm 1541 that has a first end portion or fixed end 1541a and a second end portion or free end 1541b. First end portion 1541a may be connected to and extend from a corresponding arm bend feature 1553 which may be connected to and extend from a base section 1550. Arm bend features 1553 and base section 1550 are similar to arm bend features 153 and base section 150 of gripping device 114, which is described above.

Elongate arm 1541 may be similar to elongate arm 141 in that it may include opposing side edge 1542 each forming a plurality of frictional elements 1545 (only one of which is referenced for case of illustration), such as by cutting and bending the base material forming proximal elements 1540, for example. As shown in FIG. 26B, the formation of each frictional element 1545 from a side edge 1542 may form one or more corresponding notches or openings. For example, a first notch or outer notch 1543a (also referred to herein as first opening or inner opening) may extend inwardly from a side edge in a direction toward a longitudinal axis LA of elongate arm 1541, and a second notch or inner notch 1543b (also referred to herein as second opening or inner opening) may intersect first notch 1543a and extend in a direction along longitudinal axis LA. First notch 1543a may be angled relative to longitudinal axis LA or may be perpendicular to longitudinal axis LA, and second notch 1543b may be parallel (+/−5 degrees) relative to longitudinal axis LA or may be angled relative to longitudinal axis LA, for example. Such notches 1543a, 1543b may together form the shape of a frictional element 1545 which may be bent in a distal direction such that it extends distally from a distal surface 1544b of proximal element 1540. Additionally, each frictional element 1545 may terminate at a prong 1545P or a plurality of prongs which may be configured for atraumatic frictional engagement.

Each proximal element 1540 may include a plurality of frictional elements 1545 which may be arranged in rows of one or more frictional elements 1545. For example, proximal element 1540 may include one to six rows of frictional elements 1545, and each row may include one to four frictional elements 1545 aligned in a transverse direction. In the embodiment depicted, each proximal element 1540 includes six rows of frictional elements 1545 with each row having two frictional elements 1545 formed from opposing side edges 1542 and each defining first and second notches 1543a, 1543b.

As mentioned above, tissue may become snagged by one of frictional elements 1545 or within one of the notches 1543a, 1543b between them during a TEER procedure. To help prevent or mitigate tissue from becoming snagged, proximal element 1540 may include a pair of guard rails or bumper rails 1548. Guard rails 1548 are similar to guard rails 1048 in that they may each be connected to first end portion 1541a and second end portion 1541b and extend therebetween and extend beyond all rows of frictional elements 1545 in the proximal-distal direction. Additionally, guard rails 1548 may be laterally offset from side edges 1542 of elongate arm 1541 so as to form elongate openings 1547 therebetween, as best shown in FIG. 26B. Thus, guard rails 1548 may collectively define a width wider than a width of elongate arm 1541.

However, unlike guard rails 1048, guard rails 1548 may be offset in a distal direction relative to elongate arm 1541. In this regard, each guard rail 1548 may include downwardly extending portions 1548b or connection portions and an axially extending portion 1548a. In other words, each guard rail 1548 may be connected to first and second end portions 1541a, 1541b via connection portions 1548b which may extend downwardly or distally relative to elongate arm 1541, and axially extending portion 1548a may be connected to and extend between connection portions 1548b such that axially extending portion 1548a is disposed distal of distal surface 1544b of elongate arm 1541, as best shown in FIG. 26D. As also shown in FIG. 26D, elongate arm 1541 may define a first reference plane REF1, and each guard rail 1548 may define a second reference plane REF2 offset from first reference plane REF1. In one example, guard rails 1548 may be offset from elongate arm 1541 such that guard rails 1548 are positioned at or below frictional elements 1545, particularly the prongs 1545P thereof, as shown. Thus, guard rails 1548 may help provide protection below and at the sides of proximal element 1540 to prevent tissue from becoming inadvertently caught or snagged by frictional elements 1545 and/or notches 1543a, 1543b in elongate arm 1541.

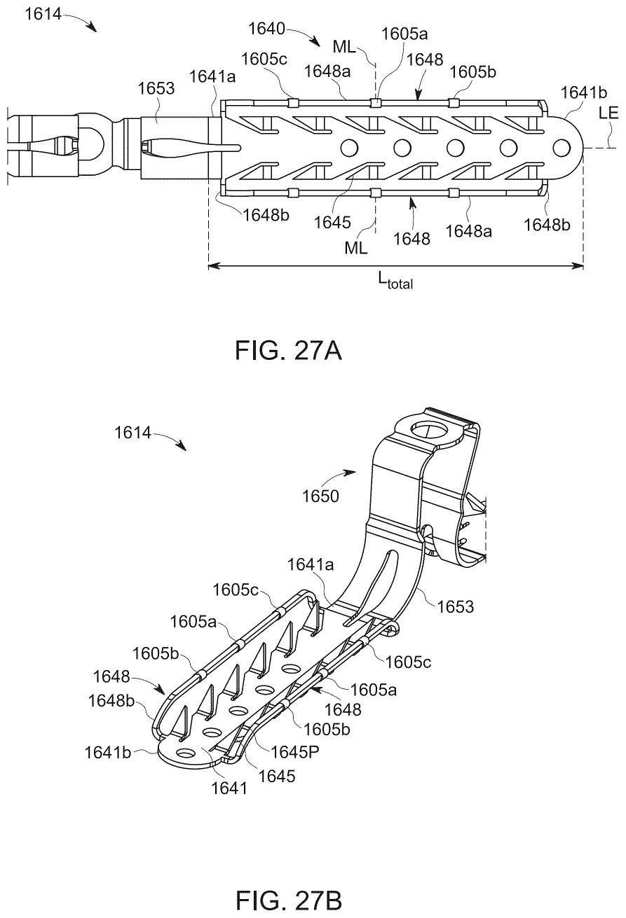

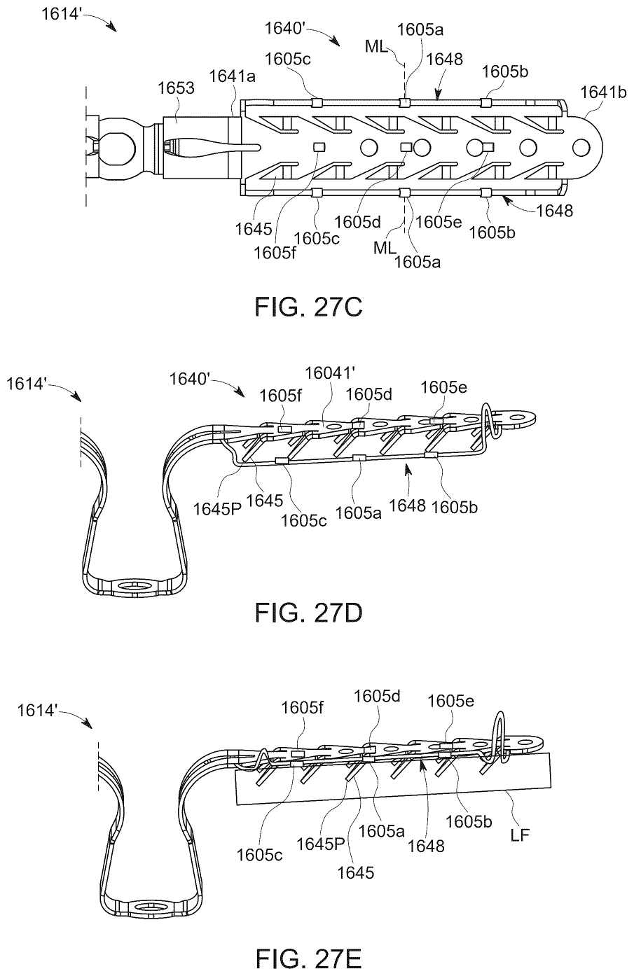

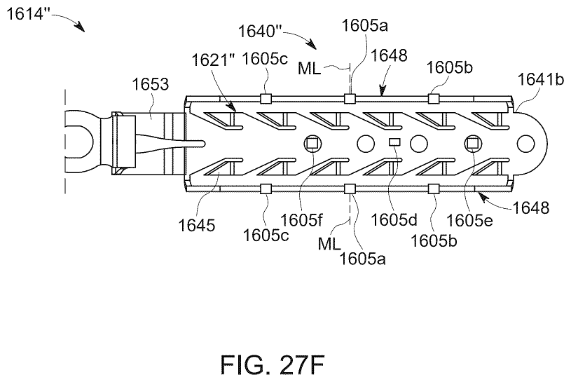

Additionally, guard rails 1548 may be flexible and may have a first configuration and a second configuration. In the first configuration or relaxed configuration, guard rails 1548 may be offset distally relative to elongate arm 1540 as described above and as shown in FIG. 26D. When proximal element 1540 engages tissue, guard rails 1548 may move proximally to the second configuration, which is depicted in FIG. 26E. In the second configuration or engagement configuration, guard rails 1548 may be positioned closer to elongate arm 1541 in the proximal-distal direction than in the first configuration. Thus, guard rails 1548 may be biased toward the first configuration. Such bias may help provide enhanced fixation of tissue as guard rails 1548, when in the second configuration, may impose pressure on tissue in addition to that provided by elongate arm 1541. The bias of guard rails 1548 may be configured such that guard rails 1548 will only move when desirable tissue engagement occurs, rather than inadvertent tissue engagement. The extent to which guard rails 1548 move relative to elongate arm 1541 may depend on the nature of the engagement with the tissue. For example, when guard rails 1548 are in the second configuration, guard rails 1548 may be flush with elongate arm 1541 such that the second reference plane REF2 defined by each guard rail 1548 is coplanar with first reference plane REF1, as shown in FIG. 26E. In other examples, guard rails 1548 may be moved to a position in a proximal-distal direction in which guard rails 1548 are located between elongate arm 1541 and prongs 1545P of frictional elements 1545. In a further example, guard rails 1548 may be moved such that second reference plane REF2 is angled relative to first reference plane REF1 which may occur when tissue is inserted partially into a space between proximal element 1540 and an opposed distal element (e.g., distal element 120) such that connection portion 1548b closest to second end portion 1541b is flexed more than connection portion 1548b closest to first end portion 1541a. Regardless of the extent to which guard rails 1548 are moved by tissue engagement, guard rails 1548 are moved in a manner that exposes some or all of frictional elements 1545 so that frictional elements 1545 can engage the tissue in a desired manner. In this regard, each guard rail 1548 may flex outwardly and upwardly at connection portions 1548b such that the width of proximal element 1540 as measured between guard rails 1548 is greater in the second configuration than in the first configuration. In other embodiments, guard rails 1548 may only flex upwardly or otherwise move proximally such that the width of proximal element 1540 defined at guard rails 1548 may be the same in the first configuration and in the second configuration (see e.g., FIG. 27E).

Guard rails 1548 may be separately connected to first and second end portions 1541a, 1541b, such as by welding, for example. Guard rails 1548 may also be formed from the same base material as elongate arm 1541 and frictional elements 1545, such as by cutting (e.g., laser cutting) the base material to form guard rails 1548. In this regard, proximal elements 1540 may be made from a memory metal material, such as nitinol, for example, which may provide for the bias of guard rails 1548 to the first configuration. A blank of such memory metal material may be cut to form frictional elements 1545 and guard rails 1548. In other embodiments, guard rails 1548 may be made from a polymeric material, such as PLLA, PLGA, PEEK, ABS, or polyurethane, for example. Guard rails 1548 may also be made from a metal material, such as titanium, cobalt-chromium, or stainless steel, for example. Further, guard rails 1548 may be made from a foam-like material which may incorporate a hydrogel, for example.

Gripping device 1514 may be utilized in conjunction with distal elements 120 of fixation device 112, as an example. Additionally, while proximal element 1540 is depicted as being connected to and extending from arm bend feature 1553 and base section 1550, which are similar to arm bend feature 153 and base section 150 of FIGS. 5A and 5C, it should be understood that proximal element 1540 can be connected to and extend from arm bend feature 260 and second arm 250 of FIGS. 6A and 6B or bend feature 260 and second arm 250′ of FIG. 6C, for example. Thus, proximal element 1540 may be adapted to couple to a center portion of a fixation device, like in fixation device 112, or adapted to couple directly to a distal element of a fixation device as has been described with respect to gripping devices 214 and 214′ of FIGS. 5A-5B and 6A-6C.

Referring now in addition to FIGS. 27A and 27B, which depict a gripping device 1614 according to another embodiment of the present disclosure. Gripping device 1614 is similar to gripping device 1514 except for differences explicitly described and/or shown and may include any features or characteristics of any other gripping device of the disclosure. Thus, like elements are accorded like reference numerals to that of gripping device 1514 but within the 1600-series of numbers, meaning elongate arm 1641 is alike to elongate arm 1541, guard rails 1658 are alike to guard rails 1548, and so on, except for differences explicitly described and/or shown.

Such differences include the addition of radiopaque indicators or markers 1605a-1615c. Radiopaque indicators 1605a-1605c may be made of a radiopaque material visible via medical imaging, such as fluoroscopy. Such radiopaque materials may include platinum, platinum-iridium or tantalum, for example. As shown, one or more radiopaque indicators 1605a-1605c may be connected to each guard rail 1648, and in particular, axial rail 1648a. For example, in the embodiment depicted, each guard rail 1648 may include a first indicator 1605a, a second indicator 1605b, and a third indicator 1605c. Proximal element 1640 may have a total length Lₜₒₜₐₗ as measured between first end portion 1641a and second end portion 1641b. First indicator 1605a may be positioned at a midline ML of proximal element 1640 to indicate a 50% tissue insertion depth. Pullout force tests have concluded there is a significant drop in retention force at 50% tissue insertion depth and that ensuring a tissue insertion depth of greater than 50% can help mitigate SLDA. Thus, first indicator 1605a may help an operator determine whether a greater than 50% leaflet insertion depth has been achieved during a TEER procedure. To further help assess tissue insertion depth, second indicator 1605b may be positioned between second end portion 1641b and midline ML, and third indicator 1605c may be positioned between midline ML and first end portion 1641a. For example, second indicator 1605b may be positioned at 25% of the total length Lₜₒₜₐₗ as measured from second end portion 1641b, and third indicator 1605c may be positioned at 75% of the total length Lₜₒₜₐₗ as also measured from second end portion 1641b. Thus, second indicator 1605b may indicate a 25% tissue insertion depth, while third indicator 1605c may indicate a 75% tissue insertion depth.

It should be understood that gripping device 1614 depicted in FIGS. 27A and 27B is but one example of the possible radiopaque indicator arrangements. Thus, in other embodiments, gripping device 1614 may have more or less radiopaque indicators 1605a-1605c than that shown. For example, each guard rail 1648 may have only have first indicator 1605a which may be positioned at the midline ML. In other embodiments, each guard rail 1648 may have only first and second indicators 1605a, 1605b, such as at the midline ML and 25% insertion depth, for example.

Additional alternative radiopaque indicators arrangements are also shown in FIGS. 27C-27F. For example, and with reference to FIGS. 27C-27E, a gripping device 1614′ like that of gripping device 1614 additionally includes one or more radiopaque indicators 1605d-1605f on elongate arm 1641′. As shown, elongate arm 1641′ may include a fourth indicator 1605d which may be aligned in the transverse direction with first indicators 1605a of guard rails 1648, a fifth indicator 1605e which may be aligned in the transverse direction with second indicators 1605b of guard rails 1648, and a sixth indicator 1605f which may be aligned in the transverse direction with third indicators 1605c of guard rails 1648. Thus, fourth indicator 1605d may be positioned at the midline ML, fifth indicator 1605e may be positioned at the 25% tissue insertion depth, and sixth indicator 1605f may be positioned at the 75% tissue insertion depth. As shown in FIG. 27D, when guard rails 1648 are in the first configuration, first, second, third, fourth, fifth, and sixth indicators 1605a-1605e may be visible via fluoroscopy which is typically directed toward gripping device 1614′ in the plane depicted. Thus, where no leaflet tissue is disposed between proximal element 1640′ and a corresponding distal element (e.g., distal element 120), all indicators 1605a-1605e may be visible. In contrast, as shown in FIG. 27E, when tissue has been inserted to a full depth, guard rails 1648 are moved to the second configuration such that they may be aligned with elongate arm 1641′ and indictors 1605a-1605e thereof may also be aligned such that they give the appearance of three indicators in the medical image thereby indicating full leaflet insertion. Where leaflet insertion is less than full depth, guard rails 1648 may be angled depending the depth of insertion. For example, where only a 25% tissue insertion depth has been achieved, fifth indicator 1605e may be closer to second indicator 1605b than fourth indicator 1605d to first indicator 1605a, and fourth indicator 1605d may be closer to first indicator 1605a than sixth indicator 1605f to third indicator 1605c. When greater than 50% insertion depth has been achieved, all indicators 1605a-1605c may align, or third and sixth indicators 1605c, 1605e may be slightly offset.

With reference to FIG. 27E, another exemplary gripping device 1614″ like that of gripping device 1614 is shown but in this example the indicators 1605d-1605e located on elongate arm 1641″ may be offset in the longitudinal direction relative to indicators 1605a-1605c on guard rails 1648. For example, fourth indicator 1605d may be positioned between first and second indicators 1605b in the longitudinal direction, fifth indicator 1605e may be positioned between second indicators 1605b and second end portion 1641b in the longitudinal direction, and sixth indicator 1605f may be positioned between first and third indicators 1605a, 1605c in the longitudinal direction. Gripping devices 1614′, 1614″ like that of gripping device 1614 may include more or less indicators on guard rails 1648 and/or elongate arm 1640′, 1640″.

Although FIGS. 27A-27F depict radiopaque indicators 1605a-1605e being connected to guard rails 1648 and/or elongate arm 1641, 1641′, 1641″, it should also be understood that such radiopaque indicators 1605a-1605e may be connected to the guard rails and/or elongate body of any of the gripping devices described herein including gripping device 1014, 1140, 1240, 1340, and those described below.

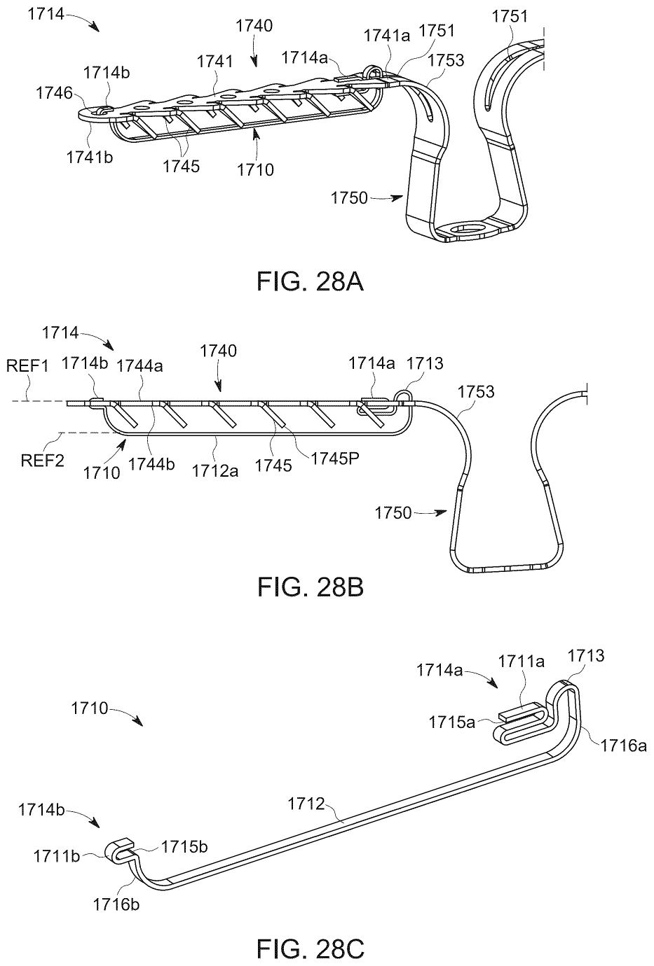

Referring now in addition to FIGS. 28A-28C, which depict a gripping device 1714 according to another embodiment of the present disclosure, which may be incorporated into any of the fixation devices of the disclosure. Gripping device 1714 is similar to gripping device 114 except for differences explicitly described and/or shown and may include any features or characteristics of any other gripping device of the disclosure. Thus, like elements are accorded like reference numerals to that of gripping device 114 but within the 1700-series of numbers, meaning base section 1750 is alike to base section 150, arm bend features 1753 are alike to arm bend features 153, proximal elements 1740 are alike to proximal elements 140, and so on, except for differences explicitly described and/or shown.

In addition to the features mentioned above, gripping device 1714 may include modular guard rails or bumper rails 1710. As shown in FIG. 28C, each modular guard rail 1710 may include an axial portion 1712 and first and second connection portions 1714a, 1714b connected to axial portion 1712. First and second connection portions 1714a, 1714b may be configured to modularly connect to an elongate arm 1741 of a proximal element 1740. In the embodiment depicted, first connection portion 1714a may be configured to connect to an opening 1751 within arm bend feature 1753. Such opening 1751 may be an elongate opening that may extend into first end portion 1741a of elongate arm 1741. For example, elongate opening 1741a may be pill-shaped, oval shaped, or have a bowling pin shape, for example, that may extend along a bend feature 1753 and may extend into first end portion 1741a of elongate arm 1741. The other openings 1746 in elongate arm 1741 to which second connection portion 1714b may be connected may be a circular, for example. However, in some embodiments both openings 1746, 1751 may be circular or both may be elongate.

First connection portion 1714a, 1714b may include first bracket or mount 1711a that may be U-shaped and may define a first slot 1715a configured to receive a portion of elongate arm 1741. First connection member 1714a may also include a leaf spring 1713 that may be connected to first bracket 1711a and may also include an intermediate member 1716a that may extend between leaf spring 1713 and axial portion 1712 of guard rail 1710. Second connection portion 1714b may have a second bracket or mount 1711b that may be U-shaped and may define a slot 1715b configured to receive a portion of elongate arm 1710. First and second brackets 1711a, 1711b may be configured such that their corresponding slots 1715a, 1715b face towards each other. Second connection member 1714b may have an intermediate member 1716b that may be connected to second bracket 1711b and may extend to axial portion 1712 of guard rail 1710. In some embodiments, second connection portion 1714b may also or may alternatively include a leaf spring 1713 like that of first connection portion 1714a.

As shown in FIGS. 28A and 28B, a modular guard rail 1710 may connect to an elongate arm 1741 of a proximal element 1740 by connecting first connection portion 1711a to opening 1751 within first end portion 1741a or first bend feature 1751 and second connection portion 1714b in opening 1746 within elongate arm 1741 which may be at second end portion 1741b such that elongate arm 1741 is received within their respective slots 1715a, 1715b. Leaf spring 1713 may extend at least partially through elongate opening 1751 within first end portion 1741a, as shown. When modular guard rail 1710 is connected to elongate arm 1741, axial portion 1712 thereof may be positioned offset in a distal direction from distal surface 1744b of elongate arm 1741 such that it may be positioned below or at the same level as prongs 1745P of frictional elements 1745 which may shield frictional elements 1745 from inadvertent engagement with tissue. Additionally, guard rail 1710 may be positioned between frictional elements 1745. For example, proximal element 1740 may include a plurality of rows of frictional elements 1745 with each row having a pair of frictional elements 1745. Guard rail 1710 may be positioned between each pair of frictional elements 1745. Thus, in some embodiments of proximal element 1740 in which each row of frictional elements 1745 includes more than two frictional elements 1745, more than one guard rail 1710 may be provided.

Guard rail 1710 may have a first configuration or relaxed configuration, which is described above. When proximal element 1740 engages tissue, leaf spring 1713 may compress, and guard rail 1710 may move proximally to a second configuration. Intermediate portion 1716a and/or intermediate portion 1716b may be flexible such that they may bend when transitioning from the first configuration to the second configuration. During this transition, leaf spring 1713 may also move further through elongate opening 1751 to take up slack allowing guard rail 1710 it to achieve a conformal profile or low-profile adjacent to distal surface 1744b of elongate arm 1741. Thus, in the second configuration or engagement configuration, guard rail 1710 may be positioned closer to distal surface of elongate arm 1741 in the proximal-distal direction than in the first configuration. Guard rail 1710 may be biased toward the first configuration. Such bias, which may at least be provided by leaf spring 1713, may help provide enhanced fixation of tissue as guard rail 1710, when in the second configuration, may impose pressure on tissue in addition to that provided by elongate arm 1741. The bias of guard rail 1710 may be configured such that guard rail 1710 will only move when desirable tissue engagement occurs, rather than inadvertent tissue engagement. The extent to which guard rail 1710 moves relative to elongate arm 1741 may depend on the nature of the engagement with the tissue. For example, when guard rails 1710 are in the second configuration, guard rail 1710 may abut distal surface 1744b of elongate arm 1740 which may expose frictional elements 1745 to engagement with tissue. However, in the first configuration, guard rail 1710 provides a guard or bumper to tissue to prevent such tissue from becoming inadvertently caught or snagged onto frictional elements 1745.

Although guard rail 1710 is described and shown as being modular, guard rail 1710 may be integral with elongate arm 1712 so as to form a monolithic structure. As such, guard rail 1710 may be made from the same base material as elongate arm 1741 and cut or otherwise machined from such base material, which may be a memory metal material. Guard rail 1710 may also be connected to elongate arm 1741 via other mechanism such as a press-fit, weld, and the like. Guard rail 1710 may also be made from other materials, such as those described above with respect to guard rail 1548. It should also be understood that guard rail 1710 may be included in any of the other gripping devices described herein.

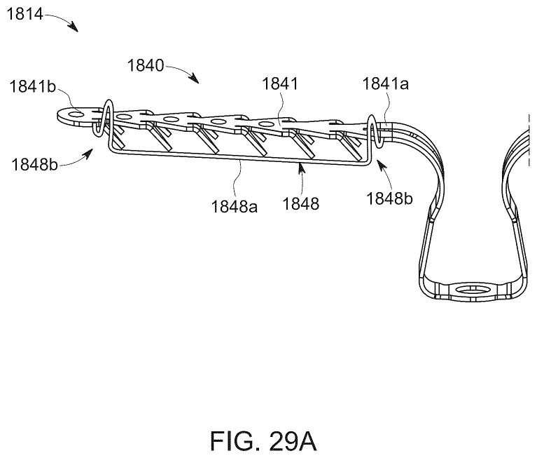

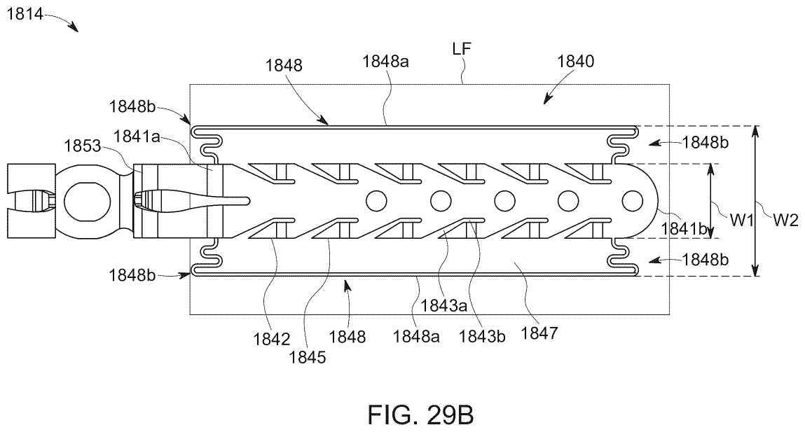

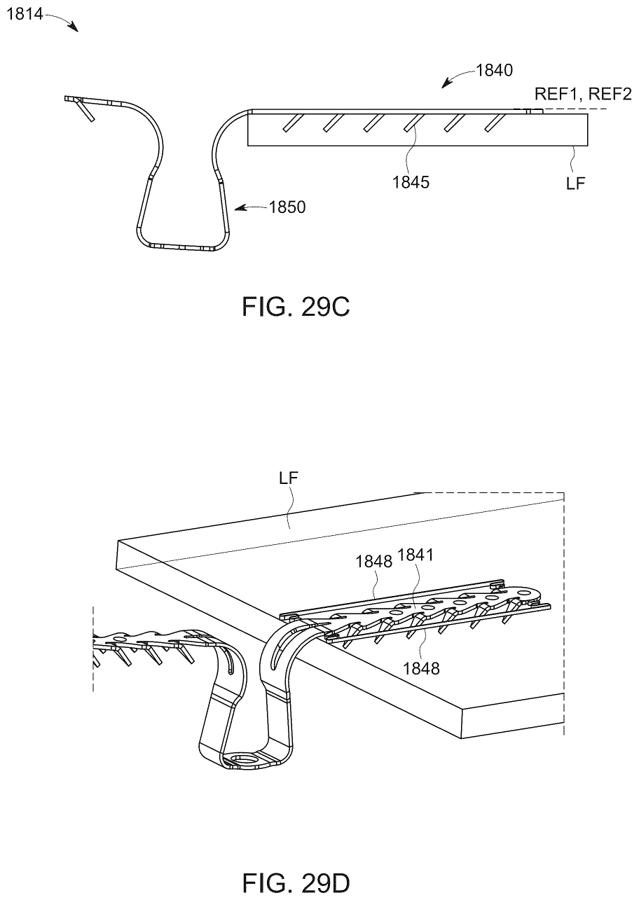

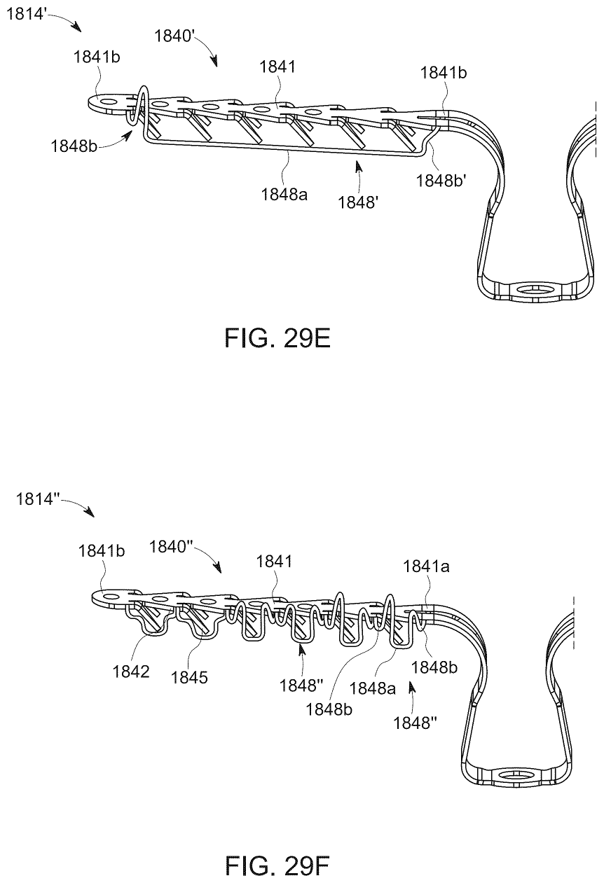

Referring now in addition to FIGS. 29A-29D, which depict a gripping device 1814 according to another embodiment of the present disclosure, which may be incorporated into any of the fixation devices of the disclosure. Gripping device 1814 is similar to gripping device 114. For case of review, like elements are accorded like reference numerals to that of gripping device 1514, but within the 1800-series of numbers. For instance, gripping device 1814 may include proximal elements 1840 with frictional elements 1845 and a pair of guard rails 1848 that have a first configuration and a second configuration. However, unlike gripping device 1514, guard rails 1848 may each have a pair of connection members 1848b may form an integral hinge that may facilitate compression and outward rotation of guard rails 1848 from the first configuration to the second configuration. Each integral hinge 1848b may be a living hinge and may have an undulating length extending between elongate arm 1841 to axial portion 1848a, for example. In other words, each integral hinge 1848b may connect to elongate arm 1841, such as at first and second end portions 1841a, 1841b, for example, and may undulate or have a wave-like or serpentine profile extending from elongate arm 1841 to axial portion 1848 of their respective guard rail 1848. In other embodiments, hinges 1848b may each be a modular hinge and may include a hinge pin and a torsion spring, for example.

In the first configuration or relaxed configuration, as shown in FIG. 29A, guard rails 1848 may be offset distally and laterally relative to elongate arm 1841, as described above with respect to gripping device 1514. When proximal element 1840 engages tissue, guard rails 1848 may move proximally to the second configuration, which is depicted in FIGS. 29C and 29D. When transitioning from the first configuration to the second configuration, guard rails 1848 may rotate outwardly so that they are positioned closer to elongate arms 1841 or adjacent to side edges 1842 of elongate arm 1840, as also described with respect to gripping device 1514.

Although gripping device 1814 is shown with guard rails 1848 that have a pair of integral hinges 1848b each connecting to first end portion 1841a and second end portion 1841b, respectively, other configurations are contemplated. For example, FIG. 29E depicts gripping device 1814′ in which each guard rail 1848′ includes a first connection portion 1848b, which may be an integral hinge, and second connection portion 1848b′ which may not be an integral hinge. Second connection portion 1848b′ may be like connection portion 1548b of guard rails 1548 and, therefore, may be flexible to facilitate the rotation of guard rails 1848 to the second configuration but may have increased stiffness relative to integral hinge 1848b to provide added bias.

In another example depicted in FIG. 29F, a gripping device 1814″ may include a pair of proximal elements 1840″ each with a plurality of guard rails connected thereto. In particular, each proximal element 1840″ of gripping device 1814″ may have a pair of guard rails 1848″ for each row of frictional elements 1845. Thus, each proximal element 1840″ may have four to six pairs of guard rails 1848″, for example. Each guard rail 1848″ may be connected to a side edge 1842 of elongate arm 1841 and may extend along only a portion of a length of elongate arm 1841. Similar to guard rails 1848, each guard rail 1848″ may include an axial portion 1848a and a pair of connection portions 1848b where each connection portion 1848b may be an integral hinge. However, in some embodiments, some or all of connection portions 1848b may not be an integral hinge like connection portions 1548b of guard rails 1548. Additionally, as mentioned above, each guard rail 1848″ may have a radiopaque indicator, like indicators 1605a-1605c, which, when associated with a row a frictional elements 1845, may help indicate which rows of frictional elements 1845 are engaged to tissue and which rows are not which may further help determine when a greater than 50% tissue insertion depth has been achieved.

Although the subject matter disclosed herein has been described with reference to particular embodiments, it is to be understood that these embodiments are merely illustrative of the principles and applications set forth in this disclosure. It is therefore to be understood that numerous modifications may be made to the exemplary embodiments and that other arrangements may be devised, such as combining one or more features of one embodiment with another embodiment or features from a plurality of embodiments, as an example. Thus, the exemplary embodiments herein are not intended to be exhaustive or to limit the disclosed subject matter to such embodiments.

1. A fixation device comprising:
a first distal element and a second distal element;
a first proximal element disposed in opposition to the first distal element and moveable relative thereto, the first proximal element having a first elongate arm having a first side edge, a second side edge, and plurality of frictional elements defining a plurality notches in the first and second side edges, the first proximal element also having a first guard rail extending adjacent to the first side edge and a second guard rail extending along the second side edge; and
a second proximal element disposed in opposition to the second distal element and moveable relative thereto, the second proximal element having a second elongate arm having a first side edge, a second side edge, and a plurality of frictional elements defining a plurality notches in the first and second side edges of the second elongate arm, the second proximal element also having a first guard rail extending adjacent to the first side edge of the second elongate arm and a second guard rail extending along the second side edge of the second elongate arm.

2. The fixation device of claim 1, wherein:
the first guard rail of the respective first and second proximal elements at least partially defines a first elongate opening in communication with the plurality of notches formed in the first side edge, and
the second guard rail of the respective first and second proximal elements at least partially defines a second elongate opening in communication with the plurality of notches formed in the second side edge.

3. The fixation device of claim 2, wherein:
the plurality of notches of the first elongate arm each include an outer notch and an inner notch, the outer notch being in communication with the inner notch, and
the plurality of notches of the second elongate arm each include an outer notch and an inner notch, the outer notch of the second proximal element being in communication with the inner notch of the second proximal element.

4. The fixation device of claim 1, wherein:
the first guard rail of the respective first and second proximal elements at least partially defines a plurality of elongate openings each being in communication with a respective one of the plurality of notches formed in the first side edge, and
the second guard rail of the respective first and second proximal elements at least partially defines a plurality of elongate openings in communication with a respective one of the plurality of notches formed in the second side edge.

5. The fixation device of claim 4, wherein the first and second guard rails of the respective first and second proximal elements are connected to the respective first and second side edges at a plurality of locations.

6. The fixation device of claim 1, wherein each frictional element of the first and second elongate arms include a prong.

7. The fixation device of claim 1, wherein the first and second elongate arms each define a first width extending in a direction transverse to a longitudinal axis thereof, and the first and second guard rails of the first and second proximal elements collectively define a second width greater than the first width.

8. The fixation device of claim 1, wherein the frictional elements of the respective first and second elongate arms are arranged in rows along a first length, and the first and second guard rails of respective first and second proximal elements extend along a second length greater than the first length.

9. The fixation device of claim 1, wherein the frictional elements of the respective first and second elongate arms are arranged in rows, and the first and second guard rails of respective first and second proximal elements extend beyond each of the rows of frictional elements in an axial direction.

10. The fixation device of claim 1, wherein the first and second elongate arms each extend from a first end portion to a second end portion, the frictional elements are each disposed between the first and second end portions, and the first and second guard rails are connected to the first and second end portions and extend therebetween.

11. A fixation device comprising:
a first distal element and a second distal element;
a first proximal element disposed in opposition to the first distal element and moveable relative thereto, the first proximal element having a plurality of frictional elements at least partially defining a plurality of openings extending through the first proximal element at first and second sides thereof, the first proximal element also having a first guard rail extending along the first side of the first proximal element and a second guard rail extending along the second side of the first proximal element; and
a second proximal element disposed in opposition to the second distal element and moveable relative thereto, the second proximal element having a plurality of frictional elements at least partially defining a plurality of openings extending through the second proximal element at first and second sides thereof, the second proximal element also having a first guard rail extending along the first side of the second proximal element and a second guard rail extending along the second side of the second proximal element.

12. The fixation device of claim 11, wherein the first and second distal elements each have a first end portion, a second end portion, and a tissue engagement surface extending between the first and second end portions of the respective first and second distal elements.

13. The fixation device of claim 11, wherein:
the first proximal element includes a first elongate arm having first and second side edges at least partially defining the first and second sides of the first proximal element, and the first and second guard rails of the first proximal element respectively flank the first and second side edges of the first elongate arm and at least partially define the first and second sides of the first proximal element; and
the second proximal element includes a second elongate arm having first and second side edges at least partially defining the first and second sides of the second proximal element, and the first and second guard rails of the second proximal element respectively flank the first and second side edges of the first elongate arm and at least partially define the first and second sides of the second proximal element.

14. The fixation device of claim 11, wherein:
the first proximal element further includes inner rails and transverse rails, the inner rails, transverse rails, and first and second guard rails of the first proximal element collectively define the openings of the first proximal element, and the frictional elements of the first proximal element each extend from a corresponding transverse rail, and
the second proximal element further includes inner rails and transverse rails, the inner rails, transverse rails, and first and second guard rails of the second proximal element collectively define the openings of the second proximal element, and the frictional elements of the second proximal element each extend from a corresponding transverse rail.

15. The fixation device of claim 14, wherein the first and second guard rails of each of the first and second proximal elements each have a varying width.

16. The fixation device of claim 11, wherein the frictional elements of the first and second proximal elements are tilted inwardly toward a longitudinal axis of the respective first and second proximal elements.

17. The fixation device of claim 11, wherein first and second proximal elements each include a fixed end and a free end, the free end being bent in a direction away from the respective first and second distal elements and including an opening configured to receive a proximal element line configured to actuate the respective first and second proximal elements between a first position and a second position.

18. A fixation device comprising:
a first distal element and a second distal element; and
a first proximal element and a second proximal element each disposed in opposition to the respective first and second distal elements and each having an elongate arm and a first guard rail and a second guard rail, the elongate arm having a plurality of frictional elements extending therefrom in a direction toward the respective first and second distal elements and arranged at intervals along a first length, and the first and second guard rails extending adjacent to the elongate arm along a second length greater than the first length.

19. The fixation device of claim 18, wherein the elongate arm includes first and second side edges, and the first and second guard rails are disposed offset from the respective first and second side edges.

20. The fixation device of claim 18, wherein the frictional elements are integral with the elongate arm and are bent toward the respective first and second distal elements so as to form openings within the first and second proximal elements that are at least partially bounded by the first and second guard rails thereof.
